SEQUENCE LISTING (290 sequences)

SEQ ID NO: 1 - protein, 36 aa, Artificial Sequence
  MeSH (protein) in anchoring cassette for plant expression
LKLLNVGVGLPVLLGGGKAYADEQGPSSSRMSYSRF

SEQ ID NO: 2 - protein, 51 aa, Artificial Sequence
  MeSH (protein) in anchoring cassette for plant expression
FQNSLATFSDTQKWGFFVSAGIVWFYLTARPGVLIGAIDAYLLAPLQLGLD

SEQ ID NO: 3 - protein, 142 aa, Artificial Sequence
  MeSH (protein) in anchoring cassette for plant expression
LKLLNVGVGLPVLLGGGKAYADEQGPSSSRMSYSRFLEYLDKGRVKKVDLFENGTSAVVEAVSPELGNRVQRVRVQFPGLSQELLQKFREKNIDFAAHNGQEETGSPLANLIGNLAFPLILIGGLFLLSRRSSGGMGGPGGG

SEQ ID NO: 4 - protein, 51 aa, Artificial Sequence
  MeSH (protein) in anchoring cassette for plant expression
METRKGEFITEEIKHASVQIMCAGVVIGLVTLVGLKFLPTRNGSPILRKMT

SEQ ID NO: 5 - protein, 21 aa, Artificial Sequence
  MeSH (protein) in anchoring cassette for plant expression
KLMGFKWIFLLSLALLQAAYY

SEQ ID NO: 6 - protein, 20 aa, Artificial Sequence
  MeSH (protein) in anchoring cassette for plant expression
VSWMSFFLLFATGAGLVYYY

SEQ ID NO: 7 - protein, 17 aa, Artificial Sequence
  MeSH (protein) in anchoring cassette for plant expression
YAVPAILLGFAGFVGFL

SEQ ID NO: 8 - protein, 21 aa, Artificial Sequence
  MeSH (protein) in anchoring cassette for plant expression
AAAVSEVTLAAADSFFPIAAL

SEQ ID NO: 9 - protein, 47 aa, Artificial Sequence
  MeSH (protein) in anchoring cassette for plant expression
AAAVSEVTLAAADSFFPIAALQQCIDMVHTFTGFEWWASIVVATILI

SEQ ID NO: 10 - protein, 24 aa, Artificial Sequence
  MeSH (protein) in anchoring cassette for plant expression
AILVVAILIVTATYQAGLSPPGGF

SEQ ID NO: 11 - protein, 27 aa, Artificial Sequence
  MeSH (protein) in anchoring cassette for plant expression
FIYAFFFIGLNGFAFVSSLYVIIIITI

SEQ ID NO: 12 - protein, 70 aa, Artificial Sequence
  MeSH (protein) in anchoring cassette for plant expression
AILVVAILIVTATYQAGLSPPGGFWQDTNDGRYGHMAGQMTMPFIYAFFFIGLNGFAFVSSLYVIIIITI

SEQ ID NO: 13 - protein, 18 aa, Artificial Sequence
  MeSH (protein) in anchoring cassette for plant expression
VVALNLFVALLCACIVLG

SEQ ID NO: 14 - protein, 48 aa, Artificial Sequence
  MeSH (protein) in anchoring cassette for plant expression
VVALNLFVALLCACIVLGHLLEENRWMNESITALLIGLGTGVTILLIS

SEQ ID NO: 15 - protein, 18 aa, Artificial Sequence
  MeSH (protein) in anchoring cassette for plant expression
VVSLNLFVALLCACIVIG

SEQ ID NO: 16 - protein, 48 aa, Artificial Sequence
  MeSH (protein) in anchoring cassette for plant expression
VVSLNLFVALLCACIVIGHLLEENRWMNESITALLIGLGTGVVILLIS

SEQ ID NO: 17 - protein, 21 aa, Artificial Sequence
  MeSH (protein) in anchoring cassette for plant expression
VIPISVFIAILCLCLVIGHLL

SEQ ID NO: 18 - protein, 47 aa, Artificial Sequence
  MeSH (protein) in anchoring cassette for plant expression
VIPISVFIAILCLCLVIGHLLEENRWVNESITAILVGAASGTVILLI

SEQ ID NO: 19 - protein, 18 aa, Artificial Sequence
  MeSH (protein) in anchoring cassette for plant expression
VVSMNLFVALLCACIVLG

SEQ ID NO: 20 - protein, 48 aa, Artificial Sequence
  MeSH (protein) in anchoring cassette for plant expression
VVSMNLFVALLCACIVLGHLLEETRWMNESITALIIGSCTGIVILLIS

SEQ ID NO: 21 - protein, 21 aa, Artificial Sequence
  MeSH (protein) in anchoring cassette for plant expression
TCLLILIFGIVLLIVIIVVLV

SEQ ID NO: 22 - protein, 21 aa, Artificial Sequence
  MeSH (protein) in anchoring cassette for plant expression
TTIVLGTTIGFLVILLAISCI

SEQ ID NO: 23 - protein, 21 aa, Artificial Sequence
  MeSH (protein) in anchoring cassette for plant expression
WTCIAIIILIIIITVVVLAVL

SEQ ID NO: 24 - protein, 21 aa, Artificial Sequence
  MeSH (protein) in anchoring cassette for plant expression
FCIDIVLLCIVLGIAAYLYNV

SEQ ID NO: 25 - protein, 20 aa, Artificial Sequence
  MeSH (protein) in anchoring cassette for plant expression
LGFMWNPLSWVMEAAALMAI

SEQ ID NO: 26 - protein, 53 aa, Artificial Sequence
  MeSH (protein) in anchoring cassette for plant expression
LGFMWNPLSWVMEAAALMAIALANGDNRPPDWQDFVGIICLLVINSTISFIEE

SEQ ID NO: 27 - protein, 21 aa, Artificial Sequence
  MeSH (protein) in anchoring cassette for plant expression
AVIAIVLCDVFGICLVGLLFT

SEQ ID NO: 28 - protein, 22 aa, Artificial Sequence
  MeSH (protein) in anchoring cassette for plant expression
VLQLVLRANLMLFYFEGFYYHI

SEQ ID NO: 29 - protein, 63 aa, Artificial Sequence
  MeSH (protein) in anchoring cassette for plant expression
VLQLVLRANLMLFYFEGFYYHISKRASGVRYVFIGKQLNQRPRYQILGVFLLIQLCILAAEGL

SEQ ID NO: 30 - protein, 21 aa, Artificial Sequence
  MeSH (protein) in anchoring cassette for plant expression
YHAILAVGVLAASGAGTAVFI

SEQ ID NO: 31 - protein, 12 aa, Artificial Sequence
  MeSH (protein) in anchoring cassette for plant expression
AIPVQVLLTGVC

SEQ ID NO: 32 - protein, 17 aa, Artificial Sequence
  MeSH (protein) in anchoring cassette for plant expression
RGKVAVSLVGLIICYAL

SEQ ID NO: 33 - protein, 21 aa, Artificial Sequence
  MeSH (protein) in anchoring cassette for plant expression
ALFAIGEVLYITRPLIYVLFI

SEQ ID NO: 34 - protein, 48 aa, Artificial Sequence
  MeSH (protein) in anchoring cassette for plant expression
ALFAIGEVLYITRPLIYVLFIRKYGVRSWIPWAISLSVDTLGMGLLAN

SEQ ID NO: 35 - protein, 18 aa, Artificial Sequence
  MeSH (protein) in anchoring cassette for plant expression
ILVTTTCLGSGYLLYKLY

SEQ ID NO: 36 - protein, 18 aa, Artificial Sequence
  MeSH (protein) in anchoring cassette for plant expression
VLVTAGCLGSGYLLYKLY

SEQ ID NO: 37 - protein, 22 aa, Artificial Sequence
  MeSH (protein) in anchoring cassette for plant expression
WEGGVGLFIVSGATLLALSWAW

SEQ ID NO: 38 - protein, 21 aa, Artificial Sequence
  MeSH (protein) in anchoring cassette for plant expression
SPLFWIGVGVGLSALFSYVTS

SEQ ID NO: 39 - protein, 24 aa, Artificial Sequence
  MeSH (protein) in anchoring cassette for plant expression
ASVKILAAGVAIGLISLFSQKYFL

SEQ ID NO: 40 - protein, 25 aa, Artificial Sequence
  MeSH (protein) in anchoring cassette for plant expression
FLGRFLLGTLAAAWFVLIPIYMWIK

SEQ ID NO: 41 - protein, 22 aa, Artificial Sequence
  MeSH (protein) in anchoring cassette for plant expression
FITPGILFLYIAGWIGWVGRSY

SEQ ID NO: 42 - protein, 24 aa, Artificial Sequence
  MeSH (protein) in anchoring cassette for plant expression
FTKRGLLLKFLILGGGSLLTYVSA

SEQ ID NO: 43 - protein, 23 aa, Artificial Sequence
  MeSH (protein) in anchoring cassette for plant expression
RASLFGVGAPEALVIGVVALLVF

SEQ ID NO: 44 - protein, 23 aa, Artificial Sequence
  MeSH (protein) in anchoring cassette for plant expression
CNALFGLGVPELAVIAGVAALLF

SEQ ID NO: 45 - protein, 23 aa, Artificial Sequence
  MeSH (protein) in anchoring cassette for plant expression
TTGVVLGVIAGSSVVLLTVNFLL

SEQ ID NO: 46 - protein, 24 aa, Artificial Sequence
  MeSH (protein) in anchoring cassette for plant expression
SEDAKAAFTAVTVSLLFRSALAEP

SEQ ID NO: 47 - protein, 21 aa, Artificial Sequence
  MeSH (protein) in anchoring cassette for plant expression
VALALTALSYLWATPGVAPGF

SEQ ID NO: 48 - protein, 23 aa, Artificial Sequence
  MeSH (protein) in anchoring cassette for plant expression
EIFKIAAIMNALTLVGVAVGFVL

SEQ ID NO: 49 - protein, 23 aa, Artificial Sequence
  MeSH (protein) in anchoring cassette for plant expression
TLNLLLLGALSLPTGYMLVPYAT

SEQ ID NO: 50 - protein, 22 aa, Artificial Sequence
  MeSH (protein) in anchoring cassette for plant expression
NYFLNIIIAIGVLVLLSTLFGG

SEQ ID NO: 51 - protein, 23 aa, Artificial Sequence
  MeSH (protein) in anchoring cassette for plant expression
PTRILGYFIGAWLVLGVALSVAF

SEQ ID NO: 52 - protein, 23 aa, Artificial Sequence
  MeSH (protein) in anchoring cassette for plant expression
PILNLIGNLAFPVILIGGLFLLS

SEQ ID NO: 53 - protein, 23 aa, Artificial Sequence
  MeSH (protein) in anchoring cassette for plant expression
DDAQTVFVAIAVSLAFRYFIAEP

SEQ ID NO: 54 - protein, 25 aa, Artificial Sequence
  MeSH (protein) in anchoring cassette for plant expression
FLLSIASGGLVLTVIIGVVVGVSNF

SEQ ID NO: 55 - protein, 26 aa, Artificial Sequence
  MeSH (protein) in anchoring cassette for plant expression
LFVGRVAMIGFAASLLGEALTGKGIL

SEQ ID NO: 56 - protein, 60 aa, Artificial Sequence
  MeSH (protein) in anchoring cassette for plant expression
LFVGRVAMIGFAASLLGEALTGKGILAQLNLETGIPIYEAEPLLLFFILFTLLGAIGALG

SEQ ID NO: 57 - protein, 23 aa, Artificial Sequence
  MeSH (protein) in anchoring cassette for plant expression
ELEVIHSRWAMLGALGCTFPEIL

SEQ ID NO: 58 - protein, 27 aa, Artificial Sequence
  MeSH (protein) in anchoring cassette for plant expression
YIGWFGVLMIPTLLTATSVFIIAFIAA

SEQ ID NO: 59 - protein, 69 aa, Artificial Sequence
  MeSH (protein) in anchoring cassette for plant expression
YIGWFGVLMIPTLLTATSVFIIAFIAAPPVDIDGIREPVSGSLLYGNNIISGAIIPTSAAIGLHFYPIW

SEQ ID NO: 60 - protein, 20 aa, Artificial Sequence
  MeSH (protein) in anchoring cassette for plant expression
LLYIFPVVILGTIACNVGLA

SEQ ID NO: 61 - protein, 21 aa, Artificial Sequence
  MeSH (protein) in anchoring cassette for plant expression
IALLSIVPKDAALFFAGAFAG

SEQ ID NO: 62 - protein, 102 aa, Artificial Sequence
  MeSH (protein) in anchoring cassette for plant expression
IALLSIVPKDAALFFAGAFAGAAAKSVTAPLDRIKLLMQTHGVRAGQQSAKKAIGFIEAITLIGKEEGIKGYWKGNLPQVIRIVPYSAVQLFAYETYKKLFR

SEQ ID NO: 63 - protein, 21 aa, Artificial Sequence
  MeSH (protein) in anchoring cassette for plant expression
EDAKAAFTAVTVSILFRSALA

SEQ ID NO: 64 - protein, 21 aa, Artificial Sequence
  MeSH (protein) in anchoring cassette for plant expression
FFVSAGIVWFYLTARPGVLIG

SEQ ID NO: 65 - protein, 21 aa, Artificial Sequence
  MeSH (protein) in anchoring cassette for plant expression
LKLLNVGVGLPVLLGGGKAYA

SEQ ID NO: 66 - protein, 21 aa, Artificial Sequence
  MeSH (protein) in anchoring cassette for plant expression
ILVFGGLTWIYLTARPGVLVG

SEQ ID NO: 67 - protein, 24 aa, Artificial Sequence
  MeSH (protein) in anchoring cassette for plant expression
VWRWVVSLMLVGLLPVSTMGRFVV

SEQ ID NO: 68 - protein, 19 aa, Artificial Sequence
  MeSH (protein) in anchoring cassette for plant expression
GVLGNAAIFAISVVAMLHL

SEQ ID NO: 69 - protein, 21 aa, Artificial Sequence
  MeSH (protein) in anchoring cassette for plant expression
FLGSVAKIVLPIIGVISLLSA

SEQ ID NO: 70 - protein, 18 aa, Artificial Sequence
  MeSH (protein) in anchoring cassette for plant expression
AAWIAGTTFLILAVPLIL

SEQ ID NO: 71 - protein, 18 aa, Artificial Sequence
  MeSH (protein) in anchoring cassette for plant expression
AAWIAGTTFLILVVPLII

SEQ ID NO: 72 - protein, 18 aa, Artificial Sequence
  MeSH (protein) in anchoring cassette for plant expression
AAWIAGTTFLILVVPLII

SEQ ID NO: 73 - protein, 48 aa, Artificial Sequence
  MeSH (protein) in anchoring cassette for plant expression
FQNSLATFSDTQKWGFFVSAGIVWFYLTARPGVLIGAIDAYLLAPLQL

SEQ ID NO: 74 - protein, 45 aa, Artificial Sequence
  MeSH (protein) in anchoring cassette for plant expression
FQNSLATFSDTQKWGFFVSAGIVWFYLTARPGVLIGAIDAYLLAP

SEQ ID NO: 75 - protein, 42 aa, Artificial Sequence
  MeSH (protein) in anchoring cassette for plant expression
FQNSLATFSDTQKWGFFVSAGIVWFYLTARPGVLIGAIDAYL

SEQ ID NO: 76 - protein, 39 aa, Artificial Sequence
  MeSH (protein) in anchoring cassette for plant expression
FQNSLATFSDTQKWGFFVSAGIVWFYLTARPGVLIGAID

SEQ ID NO: 77 - protein, 36 aa, Artificial Sequence
  MeSH (protein) in anchoring cassette for plant expression
FQNSLATFSDTQKWGFFVSAGIVWFYLTARPGVLIG

SEQ ID NO: 78 - protein, 51 aa, Artificial Sequence
  MeSH (protein) in anchoring cassette for plant expression
FQRLTEGLSHMQRWEILVFGGLTWIYLTARPGVLVGAIDAYLLAPLQLGLD

SEQ ID NO: 79 - protein, 48 aa, Artificial Sequence
  MeSH (protein) in anchoring cassette for plant expression
FQRLTEGLSHMQRWEILVFGGLTWIYLTARPGVLVGAIDAYLLAPLQL

SEQ ID NO: 80 - protein, 45 aa, Artificial Sequence
  MeSH (protein) in anchoring cassette for plant expression
FQRLTEGLSHMQRWEILVFGGLTWIYLTARPGVLVGAIDAYLLAP

SEQ ID NO: 81 - protein, 42 aa, Artificial Sequence
  MeSH (protein) in anchoring cassette for plant expression
FQRLTEGLSHMQRWEILVFGGLTWIYLTARPGVLVGAIDAYL

SEQ ID NO: 82 - protein, 39 aa, Artificial Sequence
  MeSH (protein) in anchoring cassette for plant expression
FQRLTEGLSHMQRWEILVFGGLTWIYLTARPGVLVGAID

SEQ ID NO: 83 - protein, 36 aa, Artificial Sequence
  MeSH (protein) in anchoring cassette for plant expression
FQRLTEGLSHMQRWEILVFGGLTWIYLTARPGVLVG

SEQ ID NO: 84 - DNA, 108 bp, Artificial Sequence
  MeSH DNA in anchoring cassette for plant expression
cttaagcttcttaacgttggagttggacttccagttcttcttggaggaggaaaggcttatgctgatgaacaaggaccatcttcttctagaatgtcttattctagattt

SEQ ID NO: 85 - DNA, 153 bp, Artificial Sequence
  MeSH DNA in anchoring cassette for plant expression
tttcaaaattctcttgctactttttctgatactcaaaagtggggattttttgtttctgctggaattgtttggttttatcttactgctagacctggagttcttattggagctattgatgcttatcttcttgctcctcttcaacttggacttgat

SEQ ID NO: 86 - DNA, 426 bp, Artificial Sequence
  MeSH DNA in anchoring cassette for plant expression
cttaagcttcttaacgttggagttggacttccagttcttcttggaggaggaaaggcttatgctgatgaacaaggaccatcttcttctagaatgtcttattctagatttcttgaatatcttgataagggaagagttaagaaggttgatctttttgaaaacggaacttctgctgttgttgaagctgtttctccagaacttggaaacagagttcaaagagttagagttcaatttccaggactttctcaagaacttcttcaaaagtttagagaaaagaacattgattttgctgctcataacggacaagaagaaactggatctccacttgctaaccttattggaaaccttgcttttccacttattcttattggaggactttttcttctttctagaagatcttctggaggaatgggaggaccaggaggagga

SEQ ID NO: 87 - DNA, 204 bp, Artificial Sequence
  MeSH DNA in anchoring cassette for plant expression
atggaaactgttgtttctagagctgttcttgttctttgcactccacatccaactactactcatccatttactaagccaaacaagcttcttagatcttctctttctagaggagctgcttctctttctgctacttctaagtgggctgaaagacttattgctgattttcaatttcttggagatgctgctgcttctacttctacttct

SEQ ID NO: 88 - DNA, 63 bp, Artificial Sequence
  MeSH DNA in anchoring cassette for plant expression
aaactgatgggtttcaaatggatattcctactctctctcgctcttctccaagctgcttactac

SEQ ID NO: 89 - DNA, 60 bp, Artificial Sequence
  MeSH DNA in anchoring cassette for plant expression
gtttcatggatgagctttttcttgctgtttgctactggagctggattggtctactattat

SEQ ID NO: 90 - DNA, 51 bp, Artificial Sequence
  MeSH DNA in anchoring cassette for plant expression
tatgctgttcctgcgattctacttggttttgccggattcgtcggctttctt

SEQ ID NO: 91 - DNA, 63 bp, Artificial Sequence
  MeSH DNA in anchoring cassette for plant expression
gctgctgctgtcagtgaagttacacttgcagctgctgactctttcttccctatcgcggccctt

SEQ ID NO: 92 - DNA, 141 bp, Artificial Sequence
  MeSH DNA in anchoring cassette for plant expression
gctgctgctgtcagtgaagttacacttgcagctgctgactctttcttccctatcgcggcccttcagcagtgcattgatatggtgcatacattcacaggctttgagtggtgggcatccattgttgtagcaaccattttgatc

SEQ ID NO: 93 - DNA, 72 bp, Artificial Sequence
  MeSH DNA in anchoring cassette for plant expression
gcaatacttgtggtggctatactgatagtaacagctacataccaggctggtctcagtcctcccggcggattt

SEQ ID NO: 94 - DNA, 81 bp, Artificial Sequence
  MeSH DNA in anchoring cassette for plant expression
ttcatctatgcctttttctttattggcctcaacggatttgccttcgtatcatctctatatgtgataataatcatcacaatt

SEQ ID NO: 95 - DNA, 210 bp, Artificial Sequence
  MeSH DNA in anchoring cassette for plant expression
gcaatacttgtggtggctatactgatagtaacagctacataccaggctggtctcagtcctcccggcggattttggcaggatacaaatgatggcaggtatggtcacatggctgggcagatgactatgccgttcatctatgcctttttctttattggcctcaacggatttgccttcgtatcatctctatatgtgataataatcatcacaatt

SEQ ID NO: 96 - DNA, 54 bp, Artificial Sequence
  MeSH DNA in anchoring cassette for plant expression
gtggttgcgttgaatctctttgttgcacttctttgtgcttgtattgttcttggt

SEQ ID NO: 97 - DNA, 144 bp, Artificial Sequence
  MeSH DNA in anchoring cassette for plant expression
gtggttgcgttgaatctctttgttgcacttctttgtgcttgtattgttcttggtcatcttttggaagagaatagatggatgaacgaatccatcaccgccttgttgattgggctaggcactggtgttaccattttgttgattagt

SEQ ID NO: 98 - DNA, 54 bp, Artificial Sequence
  MeSH DNA in anchoring cassette for plant expression
gtcgtttcacttaatctctttgttgcccttctatgtgcttgtatcgtcattggc

SEQ ID NO: 99 - DNA, 144 bp, Artificial Sequence
  MeSH DNA in anchoring cassette for plant expression
gtcgtttcacttaatctctttgttgcccttctatgtgcttgtatcgtcattggccatcttttggaggagaatcgatggatgaacgaatccatcactgctttattgattgggcttggcactggtgtcgtcatattgttgattagt

SEQ ID NO: 100 - DNA, 63 bp, Artificial Sequence
  MeSH DNA in anchoring cassette for plant expression
gtgataccaatctcagtgttcatcgccattctctgtctatgtttagttatcggccacttgctt

SEQ ID NO: 101 - DNA, 141 bp, Artificial Sequence
  MeSH DNA in anchoring cassette for plant expression
gtgataccaatctcagtgttcatcgccattctctgtctatgtttagttatcggccacttgcttgaagagaatcgatgggttaatgaatctattaccgccattttagtaggagcagcatcaggaacagtgatcttacttatt

SEQ ID NO: 102 - DNA, 54 bp, Artificial Sequence
  MeSH DNA in anchoring cassette for plant expression
gtcgtctccatgaatttgttcgtagctttgctttgtgcttgcatcgtgcttggt

SEQ ID NO: 103 - DNA, 144 bp, Artificial Sequence
  MeSH DNA in anchoring cassette for plant expression
gtcgtctccatgaatttgttcgtagctttgctttgtgcttgcatcgtgcttggtcacttgcttgaggagactcggtggatgaacgagtcaatcactgctcttatcattggttcgtgtactgggattgtgatcttgcttataagt

SEQ ID NO: 104 - DNA, 63 bp, Artificial Sequence
  MeSH DNA in anchoring cassette for plant expression
acatgcctacttattctcatttttgggattgttctactcattgtcatcatcgttgtcttggtc

SEQ ID NO: 105 - DNA, 63 bp, Artificial Sequence
  MeSH DNA in anchoring cassette for plant expression
actacaattgttcttggaaccactatcggcttcttggtcattctgcttgccattagctgtata

SEQ ID NO: 106 - DNA, 63 bp, Artificial Sequence
  MeSH DNA in anchoring cassette for plant expression
tggacatgtattgccattattattctcatcatcatcataactgttgtggttcttgctgtttta

SEQ ID NO: 107 - DNA, 63 bp, Artificial Sequence
  MeSH DNA in anchoring cassette for plant expression
ttctgtatcgatattgttttgttgtgtattgttctgggtatcgctgcatacttatacaatgta

SEQ ID NO: 108 - DNA, 60 bp, Artificial Sequence
  MeSH DNA in anchoring cassette for plant expression
ctggggttcatgtggaatccgctttcatgggttatggaagctgcagctctcatggccatt

SEQ ID NO: 109 - DNA, 159 bp, Artificial Sequence
  MeSH DNA in anchoring cassette for plant expression
ctggggttcatgtggaatccgctttcatgggttatggaagctgcagctctcatggccattgctttggctaatggtgataatcgacctccggattggcaagattttgtgggtattatctgtctgcttgttatcaactccacaatcagtttcattgaagaa

SEQ ID NO: 110 - DNA, 63 bp, Artificial Sequence
  MeSH DNA in anchoring cassette for plant expression
gccgtcattgccattgttctctgcgacgtgtttggtatctgccttgtgggtttgcttttcact

SEQ ID NO: 111 - DNA, 66 bp, Artificial Sequence
  MeSH DNA in anchoring cassette for plant expression
gtcttacaactggttttgcgtgccaatctgatgctcttctactttgaaggtttttattatcatata

SEQ ID NO: 112 - DNA, 189 bp, Artificial Sequence
  MeSH DNA in anchoring cassette for plant expression
gtcttacaactggttttgcgtgccaatctgatgctcttctactttgaaggtttttattatcatatatcgaaacgtgcatccggggttcgttatgttttcataggaaagcaactgaatcagagacctagataccaaattcttggggttttccttctaatccaattgtgcatccttgctgctgagggcttg

SEQ ID NO: 113 - DNA, 63 bp, Artificial Sequence
  MeSH DNA in anchoring cassette for plant expression
taccatgctattcttgctgttggagtacttgcagcctctggtgccggtacagctgtttttatt

SEQ ID NO: 114 - DNA, 36 bp, Artificial Sequence
  MeSH DNA in anchoring cassette for plant expression
gctattcctgttcaagttcttcttactggagtatgc

SEQ ID NO: 115 - DNA, 51 bp, Artificial Sequence
  MeSH DNA in anchoring cassette for plant expression
aggggaaaggttgccgtaagccttgttggattaatcatatgttatgctttg

SEQ ID NO: 116 - DNA, 63 bp, Artificial Sequence
  MeSH DNA in anchoring cassette for plant expression
gcgttgtttgcgattggtgaggttctttacataacgagaccgctcatttacgttcttttcatc

SEQ ID NO: 117 - DNA, 144 bp, Artificial Sequence
  MeSH DNA in anchoring cassette for plant expression
gcgttgtttgcgattggtgaggttctttacataacgagaccgctcatttacgttcttttcatcagaaaatatggagtccgatcttggattccttgggctatatcgctttctgtggacacactggggatgggtcttcttgcaaat

SEQ ID NO: 118 - DNA, 54 bp, Artificial Sequence
  MeSH DNA in anchoring cassette for plant expression
attttggtgacgacgacttgtttgggaagtgggtatttgctctataaactatac

SEQ ID NO: 119 - DNA, 54 bp, Artificial Sequence
  MeSH DNA in anchoring cassette for plant expression
gtcttggtgacggctggttgtttgggaagtgggtatttactctacaaactatac

SEQ ID NO: 120 - DNA, 66 bp, Artificial Sequence
  MeSH DNA in anchoring cassette for plant expression
tgggaaggtggtgttggtttgtttattgtctctggagctactcttcttgctcttagctgggcttgg

SEQ ID NO: 121 - DNA, 63 bp, Artificial Sequence
  MeSH DNA in anchoring cassette for plant expression
tcaccacttttctggattggtgttggtgttggtctatcagctttgttctcatatgtaacttca

SEQ ID NO: 122 - DNA, 72 bp, Artificial Sequence
  MeSH DNA in anchoring cassette for plant expression
gcaagtgtgaagatcctagctgctggtgtggcaattggactgatttcactgttcagccagaagtattttctt

SEQ ID NO: 123 - DNA, 75 bp, Artificial Sequence
  MeSH DNA in anchoring cassette for plant expression
ttcttgggacgcttcctcctgggaactctagcagctgcctggtttgtgttaatccctatctacatgtggatcaag

SEQ ID NO: 124 - DNA, 66 bp, Artificial Sequence
  MeSH DNA in anchoring cassette for plant expression
ttcattactccagggattcttttcctctacattgctggatggatcgggtgggttggaagaagctac

SEQ ID NO: 125 - DNA, 72 bp, Artificial Sequence
  MeSH DNA in anchoring cassette for plant expression
ttcacaaagagaggtttgcttctcaaatttttgattcttggaggaggctctttgcttacatatgttagtgcg

SEQ ID NO: 126 - DNA, 69 bp, Artificial Sequence
  MeSH DNA in anchoring cassette for plant expression
cgtgcgtctctgtttggtgttggagcacctgaggctttggttattggtgttgttgctttgttagtcttt

SEQ ID NO: 127 - DNA, 69 bp, Artificial Sequence
  MeSH DNA in anchoring cassette for plant expression
tgtaatgctttgtttggtctcggtgtgcctgagcttgctgtgattgctggcgtcgctgcgttgcttttt

SEQ ID NO: 128 - DNA, 69 bp, Artificial Sequence
  MeSH DNA in anchoring cassette for plant expression
actaccggcgtcgtgctcggtgttatcgctggctctagcgtcgtcttgctcactgtcaatttccttttg

SEQ ID NO: 129 - DNA, 72 bp, Artificial Sequence
  MeSH DNA in anchoring cassette for plant expression
tcggaggatgctaaggctgctttcacggcggttactgtttctctccttttccgatcggctttggccgagcca

SEQ ID NO: 130 - DNA, 63 bp, Artificial Sequence
  MeSH DNA in anchoring cassette for plant expression
gtagctttagctctcactgctctttcgtatctatgggcaacacctggtgttgctcctgggttt

SEQ ID NO: 131 - DNA, 69 bp, Artificial Sequence
  MeSH DNA in anchoring cassette for plant expression
gagattttcaagattgcagcaatcatgaacgctcttactcttgttggtgttgcagttggattcgttctt

SEQ ID NO: 132 - DNA, 69 bp, Artificial Sequence
  MeSH DNA in anchoring cassette for plant expression
actttgaatcttcttcttcttggggctctttctctacctactggctacatgcttgtcccttacgctacc

SEQ ID NO: 133 - DNA, 66 bp, Artificial Sequence
  MeSH DNA in anchoring cassette for plant expression
aattacttcctcaacatcataattgccattggcgttttggtcctcttgtccactctctttggtggt

SEQ ID NO: 134 - DNA, 69 bp, Artificial Sequence
  MeSH DNA in anchoring cassette for plant expression
ccaacgaggattttgggatacttcattggagcatggcttgtccttggtgttgctctttcggttgcgttc

SEQ ID NO: 135 - DNA, 69 bp, Artificial Sequence
  MeSH DNA in anchoring cassette for plant expression
ccaatactcaacttgattgggaatcttgcattccctgttattttgattggcggtttgttccttctctcg

SEQ ID NO: 136 - DNA, 69 bp, Artificial Sequence
  MeSH DNA in anchoring cassette for plant expression
gatgatgcacagactgtgtttgtggctatagctgtatcgttggcgtttcgttattttatcgcagaacca

SEQ ID NO: 137 - DNA, 75 bp, Artificial Sequence
  MeSH DNA in anchoring cassette for plant expression
ttcttgctcagcattgcttctggtggcctcgtcctcactgtcatcattggtgtcgtcgtcggcgtctccaacttt

SEQ ID NO: 138 - DNA, 78 bp, Artificial Sequence
  MeSH DNA in anchoring cassette for plant expression
ctattcgttggtcgtgttgctatgatcggtttcgctgcatcgttgcttggtgaggcgttgacgggaaaagggatatta

SEQ ID NO: 139 - DNA, 180 bp, Artificial Sequence
  MeSH DNA in anchoring cassette for plant expression
ctattcgttggtcgtgttgctatgatcggtttcgctgcatcgttgcttggtgaggcgttgacgggaaaagggatattagctcagctgaatctggagacagggataccgatttacgaagcagagccattgcttctcttcttcatcttgttcactctgttgggagccattggagctctcgga

SEQ ID NO: 140 - DNA, 69 bp, Artificial Sequence
  MeSH DNA in anchoring cassette for plant expression
gagctcgaagtgatccacagtagatgggcaatgttgggagctttaggctgcaccttccctgaaattctc

SEQ ID NO: 141 - DNA, 81 bp, Artificial Sequence
  MeSH DNA in anchoring cassette for plant expression
tacattggatggtttggtgttttgatgatccctaccttattgaccgcaacttctgtttttattatcgcattcattgctgct

SEQ ID NO: 142 - DNA, 207 bp, Artificial Sequence
  MeSH DNA in anchoring cassette for plant expression
tacattggatggtttggtgttttgatgatccctaccttattgaccgcaacttctgtttttattatcgcattcattgctgctcctccagtagatattgatggtattcgtgaacctgtttctggatctcttctttacggaaacaatattatttccggtgccattattcctacttctgcagctattggattgcatttttacccaatctgg

SEQ ID NO: 143 - DNA, 60 bp, Artificial Sequence
  MeSH DNA in anchoring cassette for plant expression
cttttatatatttttccagtagttattcttggtaccattgcctgtaacgtaggcttagcg

SEQ ID NO: 144 - DNA, 63 bp, Artificial Sequence
  MeSH DNA in anchoring cassette for plant expression
attgctttactgtcgatagtaccgaaagacgccgcactattcttcgccggagcgttcgccgga

SEQ ID NO: 145 - DNA, 306 bp, Artificial Sequence
  MeSH DNA in anchoring cassette for plant expression
attgctttactgtcgatagtaccgaaagacgccgcactattcttcgccggagcgttcgccggagccgccgcaaagtcagtgacggcaccgcttgaccgaataaagctcctaatgcagacacatggtgttcgagctgggcaacaaagtgctaagaaggctattggtttcattgaggccattactcttatcggaaaggaagaaggtattaaaggttattggaaaggaaatctacctcaggtgataaggattgtaccttatagcgcagtccagttgtttgcatatgaaacatacaagaaactcttcagg

SEQ ID NO: 146 - DNA, 63 bp, Artificial Sequence
  MeSH DNA in anchoring cassette for plant expression
gaggatgctaaggctgctttcacggcggttactgtttctatcctttttagatcagctcttgcg

SEQ ID NO: 147 - DNA, 63 bp, Artificial Sequence
  MeSH DNA in anchoring cassette for plant expression
ttctttgtctccgccggaatcgtctggttttacctcacggcgaggcctggtgttctcatcggc

SEQ ID NO: 148 - DNA, 63 bp, Artificial Sequence
  MeSH DNA in anchoring cassette for plant expression
ctgaaattgttgaatgtgggagttggcttacctgtattattaggaggtgggaaagcgtatgct

SEQ ID NO: 149 - DNA, 63 bp, Artificial Sequence
  MeSH DNA in anchoring cassette for plant expression
atcctcgtgtttggaggcttgacgtggatttacctgactgcaagaccaggtgttcttgttggt

SEQ ID NO: 150 - DNA, 72 bp, Artificial Sequence
  MeSH DNA in anchoring cassette for plant expression
gtttggagatgggtggtgagtttgatgttagtggggttactgcccgtgtcaacaatgggtcgattcgtggtg

SEQ ID NO: 151 - DNA, 57 bp, Artificial Sequence
  MeSH DNA in anchoring cassette for plant expression
ggcgtacttggaaacgctgctatatttgccataagcgtggtcgctatgctacatctt

SEQ ID NO: 152 - DNA, 63 bp, Artificial Sequence
  MeSH DNA in anchoring cassette for plant expression
ttcttaggctctgtcgcaaagatcgtgcttccaatcatcggtgttatctctctattgtctgca

SEQ ID NO: 153 - DNA, 54 bp, Artificial Sequence
  MeSH DNA in anchoring cassette for plant expression
gcggcgtggatcgccggtacgacgtttctcatccttgccgtgcctttgatctta

SEQ ID NO: 154 - DNA, 54 bp, Artificial Sequence
  MeSH DNA in anchoring cassette for plant expression
gcagcgtggatcgctgggacaacttttctcatacttgttgttccattgatcatc

SEQ ID NO: 155 - DNA, 54 bp, Artificial Sequence; identical to SEQ ID NO: 154
  MeSH DNA in anchoring cassette for plant expression

SEQ ID NO: 156 - DNA, 144 bp, Artificial Sequence
  MeSH DNA in anchoring cassette for plant expression
tttcagaactccctcgccacattttcggatacccagaaatgggggttctttgtctccgccggaatcgtctggttttacctcacggcgaggcctggtgttctcatcggcgccattgacgcctaccttctcgctcccctccagctt

SEQ ID NO: 157 - DNA, 135 bp, Artificial Sequence
  MeSH DNA in anchoring cassette for plant expression
tttcagaactccctcgccacattttcggatacccagaaatgggggttctttgtctccgccggaatcgtctggttttacctcacggcgaggcctggtgttctcatcggcgccattgacgcctaccttctcgctccc

SEQ ID NO: 158 - DNA, 126 bp, Artificial Sequence
  MeSH DNA in anchoring cassette for plant expression
tttcagaactccctcgccacattttcggatacccagaaatgggggttctttgtctccgccggaatcgtctggttttacctcacggcgaggcctggtgttctcatcggcgccattgacgcctacctt

SEQ ID NO: 159 - DNA, 117 bp, Artificial Sequence
  MeSH DNA in anchoring cassette for plant expression
tttcagaactccctcgccacattttcggatacccagaaatgggggttctttgtctccgccggaatcgtctggttttacctcacggcgaggcctggtgttctcatcggcgccattgac

SEQ ID NO: 160 - DNA, 108 bp, Artificial Sequence
  MeSH DNA in anchoring cassette for plant expression
tttcagaactccctcgccacattttcggatacccagaaatgggggttctttgtctccgccggaatcgtctggttttacctcacggcgaggcctggtgttctcatcggc

SEQ ID NO: 161 - DNA, 153 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggtgccattgatgcgtaccttttggcacctcttcaactgggtttggac

SEQ ID NO: 162 - DNA, 144 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggtgccattgatgcgtaccttttggcacctcttcaactg

SEQ ID NO: 163 - DNA, 135 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggtgccattgatgcgtaccttttggcacct

SEQ ID NO: 164 - DNA, 126 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggtgccattgatgcgtacctt

SEQ ID NO: 165 - DNA, 117 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggtgccattgat

SEQ ID NO: 166 - DNA, 108 bp, Artificial Sequence
  MeSH DNA in anchoring cassette for plant expression
ttccagagactgaccgaaggcttatcccacatgcagaggtgggagatcctcgtgtttggaggcttgacgtggatttacctgactgcaagaccaggtgttcttgttggt

SEQ ID NO: 167 - DNA, 372 bp, Artificial Sequence
  Targeting signal in anchoring cassette for plant expression
atggcttccatctcaagctctgttgcaactgtttcaagaactgctcctgctcaagccaacatggtggctcccttcactgggttgaagagcaatgctgctttcccaacaacaaagaaggcaaatgatttctcaacccttccaagcaatggaggaagggttcaatgcatgcaagtttggccagcttatggaaacaagaagtttgaaaccctctcttatcttcctcctctctcaatggctccaactgtcatgatggcttcttcagcaactgctgttgctccctttcaagggttgaagagcactgcttctcttcctgttgcaagaagaagctcaagaagccttggaaatgtttccaatggaggaaggatcagatgc

SEQ ID NO: 168 - DNA, 192 bp, Artificial Sequence
  Targeting signal in anchoring cassette for plant expression
atggcagcaccattggcatgtcttgttgggagtggtttatctatacagagtaataaattaactcttggcaaggacgtcaatggaagatatctcttctcatatcataggctttcagcacagagtaaggagtcaaagacaatatgcataaaggcatccttggatcaaaggaagcatgaagggagaagaggattt

SEQ ID NO: 169 - DNA, 204 bp, Artificial Sequence
  Targeting signal in anchoring cassette for plant expression
atggaaacggtggtctctcgcgctgtcctcgttctctgcactccgcacccaaccacaactcaccctttcactaaacctaacaaactcctccgttcctccctcagccgcggcgccgcttccttatccgccaccagcaaatgggcggagcgcctcattgccgatttccaattcctcggcgacgccgccgcctccacttccacctcc

SEQ ID NO: 170 - DNA, 192 bp, Artificial Sequence
  Targeting signal in anchoring cassette for plant expression
atggttggaggaggagttatccagcaaattcttaggaggaagcttcactcccaatcacttgcaactccggttttgtcttggttttcttcgaagaaagctcatgaggatgctggttcttctggtgtaagagcacttgccttgttgggtgctggtgttacaggactgttgagtttttctacagtagcatctgct

SEQ ID NO: 171 - DNA, 192 bp, Artificial Sequence
  Targeting signal in anchoring cassette for plant expression
atggttggaggaggagttatccggcagcttcttagaaggaaacttcattcccaatcagtcgcaactccggtattgtcatggttgtcttcgaagaaagcgaatgaagatgctgggtctgctggtttaagagcatttgctctcatgggtgctggtattaccggattattgagtttttctactgttgcatctgct

SEQ ID NO: 172 - DNA, 60 bp, Artificial Sequence
  Targeting signal in anchoring cassette for plant expression
atggcgtctgctctatgtagaactgcaagccgtcttcgttccgttcaactttttcgtcgg

SEQ ID NO: 173 - DNA, 105 bp, Artificial Sequence
  Targeting signal in anchoring cassette for plant expression
atgcttccttgtcgccgtcttgttctgtcctgcaagaatcaagctgccagttttctccgaaggtgtggaccatcgaaacgaattcaatcagtcaattactgtaaa

SEQ ID NO: 174 - DNA, 42 bp, Artificial Sequence
  Targeting signal in anchoring cassette for plant expression
atggctttcaggcaaactttatctataaggtccaggcttttc

SEQ ID NO: 175 - DNA, 33 bp, Artificial Sequence
  Targeting signal in anchoring cassette for plant expression
atgaaattgctgcgtaaaaccgttaaagtaaaa

SEQ ID NO: 176 - DNA, 111 bp, Artificial Sequence
  Targeting signal in anchoring cassette for plant expression
atgttggattctctagtgtcgaaactgccttcgttatcgacatctgatcacgcttctgtggttgcgttgaatctctttgttgcacttctttgtgcttgtattgttcttggt

SEQ ID NO: 177 - DNA, 63 bp, Artificial Sequence
  Targeting signal in anchoring cassette for plant expression
atgacaatgttcgcctctttaacctctaaaatgctatcggtgtcaacttctgatcacgcatct

SEQ ID NO: 178 - DNA, 57 bp, Artificial Sequence
  Targeting signal in anchoring cassette for plant expression
atgagtatcggattaacagagtttgtgacgaataaactagcagctgagcatcctcag

SEQ ID NO: 179 - DNA, 66 bp, Artificial Sequence
  Targeting signal in anchoring cassette for plant expression
atggtgatcggattaagcacaatgctggaaaaaacagaggctcttttcgcttctgatcatgcatcg

SEQ ID NO: 180 - DNA, 24 bp, Artificial Sequence
  Targeting signal in anchoring cassette for plant expression
atgagtttccaagatctcgaagct

SEQ ID NO: 181 - DNA, 66 bp, Artificial Sequence
  Targeting signal in anchoring cassette for plant expression
atgaaagtgcagcgtctgttcttagtagctattttctgcctctcttatatgcagctggtcaagggg

SEQ ID NO: 182 - DNA, 3 bp, Artificial Sequence
  Targeting signal in anchoring cassette for plant expression
atg

SEQ ID NO: 183 - DNA, 183 bp, Artificial Sequence
  Targeting signal in anchoring cassette for plant expression
atgtcaggtctcgaagatatcaagaacgagaccgttgatctggaaaaaattccgattgaggaagttttccagcagctaaaatgtacaagggaaggattgacaacgcaggaaggggaagacaggattgtgatatttggccccaacaagctcgaagagaagaaggaaagcaaaattctgaagttt

SEQ ID NO: 184 - DNA, 60 bp, Artificial Sequence
  Targeting signal in anchoring cassette for plant expression
atgttggcctcgctgatcatcttcgttgctcttctctgcaatgtgactgtcataagtggc

SEQ ID NO: 185 - DNA, 42 bp, Artificial Sequence
  Targeting signal in anchoring cassette for plant expression
atggatttagtcaggggtttgtggaggaaacatagaaggaag

SEQ ID NO: 186 - DNA, 42 bp, Artificial Sequence
  Targeting signal in anchoring cassette for plant expression
atggatttcgttaggggtttttggaggaagcatagaaggaag

SEQ ID NO: 187 - DNA, 135 bp, Artificial Sequence
  Targeting signal in anchoring cassette for plant expression
atgattgggaatccagtaattcaagttccatcatcactaatgccatcatcctccatgattgcttgtcctcgagtttcacccaatggggttccttatcttccaccaaaacctagaactaggcatttagtggtcaga

SEQ ID NO: 188 - DNA, 273 bp, Artificial Sequence
  Targeting signal in anchoring cassette for plant expression
atggagaaccttaccctagtttcttgctcagcttcttctccaaagctgttaattggatgcaatttcacttcctcgctgaaaaaccctactgggttttctcgtcggactcctaatattgtcctccggtgttccaaaatatctgcctctgctcaatctcaatctccctcttcgcgtccggagaacactggagaaatcgtggttgtgaaacagagaagcaaagcttttgcaagtatattttcttcgagtcgtgatcaacagacaacttctgttgct

SEQ ID NO: 189 - DNA, 228 bp, Artificial Sequence
  Targeting signal in anchoring cassette for plant expression
atggaagctctgagtcacgtcggcattggtctctccccattccaattatgccgattaccaccggcgacgacaaagctccgacgtagccacaacacctctacaactatctgctccgccagcaaatgggccgaccgtcttctctccgacttcaatttcacctccgattcctcctcctcctccttcgccaccgccaccaccaccgccactctcgtctctccgccaccatct

SEQ ID NO: 190 - DNA, 204 bp, Artificial Sequence
  Targeting signal in anchoring cassette for plant expression
atggcctctttgatgctcaacggggccattaccttccccaaaggtttaggttcccctggttccaatttgcatgccagatcgattcctcggccgaccttactctcagttacccgaacctccacacctagactctcggtggctactagatgcagcagcagcagcgtgtcgtcttcccggccatcggcgcaacctaggttcattcag

SEQ ID NO: 191 - DNA, 180 bp, Artificial Sequence
  Targeting signal in anchoring cassette for plant expression
atgtcgctcactatcccggcgaatctcgttctcaacccgagatctaacaaatctctcactcaatccgtacctaaatcctccgcaagattcgtctgctccgatgacaaatcctcctcctccacacctcaatccatgaaagctttctccgccgccgtcgccctctcttccatcctcctctct

SEQ ID NO: 192 - DNA, 150 bp, Artificial Sequence
  Targeting signal in anchoring cassette for plant expression
atggcgtcttttgcaaccatcgccgccgtacaaccttctgccgccgtgaaaggacttggaggtagctctcttgccggagctaagctcttcatcaaaccttctcgccaaagcttcaaaaccaaatccaccagagctggtgctgtggtggct

SEQ ID NO: 193 - DNA, 246 bp, Artificial Sequence
  Targeting signal in anchoring cassette for plant expression
atggccatggcgttacagattatagcttcttcttcttcatcaccaacgattacaaaatctcaccttttctcttatcctcctctgcaatctcgttataaagcttcgaaacctaacctctcctcatggttttctcttctgggttcttctcgtttctctccttacattggtttaaagcatttgggcatctcaatctcgccgaaatcttcaaacccagagaagaagagaagatgtaagagtatgatgatt

SEQ ID NO: 194 - DNA, 177 bp, Artificial Sequence
  Targeting signal in anchoring cassette for plant expression
atggcgacatctgttgcgactctatcttctccaccaccagtatctctacctctcctctcatcatctcgctcttccttcttctccaattgcttcacagtcacgacccgaccaaacactcgttctctggtagcgattggacgcagaatccgacaagaaccaacgagaaagccactgact

SEQ ID NO: 195 - DNA, 114 bp, Artificial Sequence
  Targeting signal in anchoring cassette for plant expression
atgtcactaaccgcacaattctcgccgccggtcaccggaataaccaggagtctccgggatacaaaaccgtcactgtctaatctccgtgtatttccggtttacactgagataaga

SEQ ID NO: 196 - DNA, 204 bp, Artificial Sequence
  Targeting signal in anchoring cassette for plant expression
atggcgattagggttaccttcacctactccagctatgttgctaggagcattgcttcatccgccgggactcgtgtcggtaccggcgacgttagatcatgcttcgaaacatgggttcgtcccaggttctgcggccacaatcagataccagatattgttgataaatctcccggatccaacacatggggtccaagctcaggccctcgt

SEQ ID NO: 197 - DNA, 135 bp, Artificial Sequence
  Targeting signal in anchoring cassette for plant expression
atggctacaatatctccgggaggagcttacatcggaactccctcaccgtttctcggcaaaaaactcaaacctttctctctaacttcaccgattttgagttttaaaccaacggttaagctcaattccagttgtcgc

SEQ ID NO: 198 - DNA, 222 bp, Artificial Sequence
  Targeting signal in anchoring cassette for plant expression
atggcgacagcagcagcaccagcagtgatttcatggacaagatcaggcattgtgtccaaatccggacaaacccagaagaaatctgagatgaaagtttcttacataactggacttaactcatatggtggtctcaaggcacagaacaacaaggttgtctcaatgggatcaccactctgcacagaacagtgttttgctaacgttgtgatgtctctcaaaggaaga

SEQ ID NO: 199 - DNA, 150 bp, Artificial Sequence
  Targeting signal in anchoring cassette for plant expression
atggcgatgtcaagtgggttgtttgtgaagccaacgaagatgaatcatcaaatggttagaaaagagaagattggattgagaatttcttgtcaagcgtcgagtattccagcagacagagttccagatatggaaaagaggaagactttgaat

SEQ ID NO: 200 - DNA, 243 bp, Artificial Sequence
  Targeting signal in anchoring cassette for plant expression
atgtctgtttctctctccgccgcctctcacttactctgttcctccaccagagtctctctttcccccgccgtcacctcttcgtcctcgtcccctgtcgttgctctttcttcctctacatcgccacattctcttggaagcgtcgcatcttcttctctgtttcctcactcctccttcgtgcttcagaaaaaacatccgatcaatgggacgagcacgaggatgatttcaccaaaatgtgccgcttct

SEQ ID NO: 201 - DNA, 159 bp, Artificial Sequence
  Targeting signal in anchoring cassette for plant expression
atggcttccttatcgacgataactcaaccatctttagtccacattcccggtgaatcagtcctccatcacgtaccaagcacgtgctctttcccatggaagccaactatcaacacgaagcggattatctgttcgccggctaggaactccagcgaagtatcg

SEQ ID NO: 202 - DNA, 111 bp, Artificial Sequence
  Targeting signal in anchoring cassette for plant expression
atggctgcttcatcggcttgtcttctcggaaatggattatctgtctacacaaccaaacagaggttccaaaagcttgggcttgataggacatctaaagttacagttgtgaag

SEQ ID NO: 203 - DNA, 201 bp, Artificial Sequence
  Targeting signal in anchoring cassette for plant expression
atgatggtgatgatatctcttcacttctctactccgccgctagcttttctcaaatctgattccaattccagatttctcaagaaccctaatcctaattttatccaattcaccccaaaatcccaacttcttttcccccaacgtctcaatttcaacaccggaacaaatttaaaccggcgaactctgagttgctatgggattaag

SEQ ID NO: 204 - DNA, 105 bp, Artificial Sequence
  Targeting signal in anchoring cassette for plant expression
atggcttctacctccgcgatgtcattggtcacaccacttaaccagacccgttcgtctcctttccttaagccattacctctgaaaccatccaaggctttggttgca

SEQ ID NO: 205 - DNA, 186 bp, Artificial Sequence
  Targeting signal in anchoring cassette for plant expression
atggctcaaaccatgctgcttacttcaggcgtcaccgccggccattttttgaggaacaagagccctttggctcagcccaaagttcaccatctcttcctctctggaaactctccggttgcactaccatctaggagacaatcattcgttcctctcgctctcttcaaacccaaaaccaaagctgctcct

SEQ ID NO: 206 - DNA, 126 bp, Artificial Sequence
  Targeting signal in anchoring cassette for plant expression
atggccacttcagcaatccaacactcttctttcgctggccaaacgaccctaaagccatccaacgatctcctccgcaaaatcggagcctccaatggcggtggccgcatcatcatgcgtcgtaccgtc

SEQ ID NO: 207 - DNA, 84 bp, Artificial Sequence
  Targeting signal in anchoring cassette for plant expression
atgactgcaattttagagagacgcgaaagcgaaagcctatggggtcgcttctgtaactggataactagcactgaaaaccgtctt

SEQ ID NO: 208 - DNA, 105 bp, Artificial Sequence
  Targeting signal in anchoring cassette for plant expression
atgggagtgacaaaaaaaccagatttgaatgatcctgtattacgagctaaattagctaaaggtatgggtcacaattattacggggaacccgcatggcccaacgac

SEQ ID NO: 209 - DNA, 183 bp, Artificial Sequence
  Targeting signal in anchoring cassette for plant expression
atgggagaagagaagtctctgcttcagttccgtagttttccttcactcaagacctctgatttcgctctcaccgaagaaccttcatggaggctggagaacaacgtgtcgtcgaatcgccggagaggaaacaagagaagcggtggcgtttttaccaattttgcgtccctttccgtagcgattagg

SEQ ID NO: 210 - DNA, 156 bp, Artificial Sequence
  Targeting signal in anchoring cassette for plant expression
atggcgataagaatcactttcacttactccacccatgttgctcggaatctcgtcgggacccgtgttggtcccggtggttactgctttgaatccttggttcgtcctagattcttctcccacaagcgtgatttcgatagatctcctcggaatcgacct

SEQ ID NO: 211 - DNA, 210 bp, Artificial Sequence
  Targeting signal in anchoring cassette for plant expression
atggcctctcttctctctcccgccacccccaccgccacctccgccgcatttcattcctgctccaccgccggattctccactcccactcacatttcatcccagaacagcagcctgtccctcctcagccgccggggttgtatgatgagatgcagtttttctccgcaggacattcctgttgattccctctctcatctccctcctttccttgac

SEQ ID NO: 212 - DNA, 171 bp, Artificial Sequence
  Targeting signal in anchoring cassette for plant expression
atggcttctctgctcctccctccaactccaactgctacttcttccacagttaaacacaacaaaatccaaacgaaactgaaaccaaacagcaatgctcatgcagctacatgtaatttcttcttatcatcgaattcaaacgcaaaccaccgtttcaactctgcaaagtgccat

SEQ ID NO: 213 - DNA, 78 bp, Artificial Sequence
  Targeting signal in anchoring cassette for plant expression
atggcaacatcagctcgtagagcatacggtttcggtagagccgatgaggctacacaccctgactccattagagcaact

SEQ ID NO: 214 - DNA, 165 bp, Artificial Sequence
  Targeting signal in anchoring cassette for plant expression
atgaaagccctcacactggcactcttcttagccctttccctctatctcctccccaatcccgcccattccaggttcaatcccatccgcctccccaccacacacgaacccgcctcctctgaaactccagtactggacatcaacggcgacgaggtccgcgccggcggg

SEQ ID NO: 215 - DNA, 117 bp, Artificial Sequence
  Targeting signal in anchoring cassette for plant expression
atgaaagccctcacactggcactcttcttagccctttccctctatctcctccccaatcccgcctcctctgaaactccagtactggacatcaacggcgacgaggtccgcgccggcggg

SEQ ID NO: 216 - DNA, 72 bp, Artificial Sequence
  Targeting signal in anchoring cassette for plant expression
atgaagcagcttctgtgttaccttccatggctgcttctcctcactcttctggtttcccctttaaacgacgct

SEQ ID NO: 217 - DNA, 78 bp, Artificial Sequence
  Targeting signal in anchoring cassette for plant expression
atgggtttagtcaacgggagagcttcgttgacctttctcctcgcggcgttgaccatcatcgctatggtcgtcgaggct

SEQ ID NO: 218 - DNA, 66 bp, Artificial Sequence
  Targeting signal in anchoring cassette for plant expression
atgaagtgttggagattgtcggcgattctgtttctagggtttatgttgactagcttgtcaacggcg

SEQ ID NO: 219 - DNA, 57 bp, Artificial Sequence
  Targeting signal in anchoring cassette for plant expression
atgttatatttagttaggatagtaacagtaattggtgtggattcgaaaaagatgaag

SEQ ID NO: 220 - DNA, 216 bp, Artificial Sequence
  Targeting signal in anchoring cassette for plant expression
atgggagaaatggagatcgaagaaatcgaagctgttcttgagaaaatctgggatctacatgacaagcttagcgatgagattcacttgatttcgaagtctcactttctcaaatcggttaaaccttcaaatagatcggagaagagaaagaatcctcacggaaattccggcgaagataagcgacctggttatgttttcatcaaaggattcgccgtcgat

SEQ ID NO: 221 - DNA, 96 bp, Artificial Sequence
  Targeting signal in anchoring cassette for plant expression
atgggagaaatggagatcgaagaaatcgaagctgttcttgagaaaatctgggatctacatgacaagcttagcgatgagattcacttgatttcgaag

SEQ ID NO: 222 - DNA, 207 bp, Artificial Sequence
  Targeting signal in anchoring cassette for plant expression
atggaagacgaagaaggcatcgggttaattttagccagagccaccgagctcagactcaagatcagcgattgtatcgataactccagtaccaccgtcagcgacaacggcgatggaaacgaggatctttctcccggagaaggaagaaaaagcgagattattgggaatcaagacaaggattttgactcaatcagctctgaggatgtagat

SEQ ID NO: 223 - DNA, 144 bp, Artificial Sequence
  Targeting signal in anchoring cassette for plant expression
atggcgcctaagaaaatcggagccggcaaaggcgattcgagcatcttggcgaagatttcaaactacgatatcgtttctcagggaagacgcgccgcttgcgatgccgtctacgtgtcgaagaagctgttaaagagcaccgggaag

SEQ ID NO: 224 - DNA, 75 bp, Artificial Sequence
  Targeting signal in anchoring cassette for plant expression
atggcggcgaagagaatcggagccggtaaatctggcggcggagatccaaacatattagcgaggatttcaaactcc

SEQ ID NO: 225 - DNA, 153 bp, Artificial Sequence
  Targeting signal in anchoring cassette for plant expression
atggcggcgaagagaatcggagccggtaaatctggcggcggagatccaaacatattagcgaggatttcaaactccgagatcgtgtcccaaggaagacgcgcagcaggagatgccgtcgaagtatcgaagaaactgctccggagcaccggaaaa

SEQ ID NO: 226 - DNA, 75 bp, Artificial Sequence; identical to SEQ ID NO: 224
  Targeting signal in anchoring cassette for plant expression

SEQ ID NO: 227 - protein, 124 aa, Artificial Sequence
  Targeting signal (protein) in anchoring cassette for plant expression
MASISSSVATVSRTAPAQANMVAPFTGLKSNAAFPTTKKANDFSTLPSNGGRVQCMQVWPAYGNKKFETLSYLPPLSMAPTVMMASSATAVAPFQGLKSTASLPVARRSSRSLGNVSNGGRIRC

SEQ ID NO: 228 - protein, 64 aa, Artificial Sequence
  Targeting signal (protein) in anchoring cassette for plant expression
MAAPLACLVGSGLSIQSNKLTLGKDVNGRYLFSYHRLSAQSKESKTICIKASLDQRKHEGRRGF

SEQ ID NO: 229 - protein, 68 aa, Artificial Sequence
  Targeting signal (protein) in anchoring cassette for plant expression
METVVSRAVLVLCTPHPTTTHPFTKPNKLLRSSLSRGAASLSATSKWAERLIADFQFLGDAAASTSTS

SEQ ID NO: 230 - protein, 64 aa, Artificial Sequence
  Targeting signal (protein) in anchoring cassette for plant expression
MVGGGVIQQILRRKLHSQSLATPVLSWFSSKKAHEDAGSSGVRALALLGAGVTGLLSFSTVASA

SEQ ID NO: 231 - protein, 64 aa, Artificial Sequence
  Targeting signal (protein) in anchoring cassette for plant expression
MVGGGVIRQLLRRKLHSQSVATPVLSWLSSKKANEDAGSAGLRAFALMGAGITGLLSFSTVASA

SEQ ID NO: 232 - protein, 20 aa, Artificial Sequence
  Targeting signal (protein) in anchoring cassette for plant expression
MASALCRTASRLRSVQLFRR

SEQ ID NO: 233 - protein, 35 aa, Artificial Sequence
  Targeting signal (protein) in anchoring cassette for plant expression
MLPCRRLVLSCKNQAASFLRRCGPSKRIQSVNYCK

SEQ ID NO: 234 - protein, 14 aa, Artificial Sequence
  Targeting signal (protein) in anchoring cassette for plant expression
MAFRQTLSIRSRLF

SEQ ID NO: 235 - protein, 11 aa, Artificial Sequence
  Targeting signal (protein) in anchoring cassette for plant expression
MKLLRKTVKVK

SEQ ID NO: 236 - protein, 37 aa, Artificial Sequence
  Targeting signal (protein) in anchoring cassette for plant expression
MLDSLVSKLPSLSTSDHASVVALNLFVALLCACIVLG

SEQ ID NO: 237 - protein, 21 aa, Artificial Sequence
  Targeting signal (protein) in anchoring cassette for plant expression
MTMFASLTSKMLSVSTSDHAS

SEQ ID NO: 238 - protein, 19 aa, Artificial Sequence
  Targeting signal (protein) in anchoring cassette for plant expression
MSIGLTEFVTNKLAAEHPQ

SEQ ID NO: 239 - protein, 22 aa, Artificial Sequence
  Targeting signal (protein) in anchoring cassette for plant expression
MVIGLSTMLEKTEALFASDHAS

SEQ ID NO: 240 - protein, 8 aa, Artificial Sequence
  Targeting signal (protein) in anchoring cassette for plant expression
MSFQDLEA

SEQ ID NO: 241 - protein, 22 aa, Artificial Sequence
  Targeting signal (protein) in anchoring cassette for plant expression
MKVQRLFLVAIFCLSYMQLVKG

SEQ ID NO: 242 - protein, 1 aa, Artificial Sequence
  Targeting signal (protein) in anchoring cassette for plant expression
M

SEQ ID NO: 243 - protein, 61 aa, Artificial Sequence
  Targeting signal (protein) in anchoring cassette for plant expression
MSGLEDIKNETVDLEKIPIEEVFQQLKCTREGLTTQEGEDRIVIFGPNKLEEKKESKILKF

SEQ ID NO: 244 - protein, 20 aa, Artificial Sequence
  Targeting signal (protein) in anchoring cassette for plant expression
MLASLIIFVALLCNVTVISG

SEQ ID NO: 245 - protein, 14 aa, Artificial Sequence
  Targeting signal (protein) in anchoring cassette for plant expression
MDLVRGLWRKHRRK

SEQ ID NO: 246 - protein, 14 aa, Artificial Sequence
  Targeting signal (protein) in anchoring cassette for plant expression
MDFVRGFWRKHRRK

SEQ ID NO: 247 - protein, 45 aa, Artificial Sequence
  Targeting signal (protein) in anchoring cassette for plant expression
MIGNPVIQVPSSLMPSSSMIACPRVSPNGVPYLPPKPRTRHLVVR

SEQ ID NO: 248 - protein, 91 aa, Artificial Sequence
  Targeting signal (protein) in anchoring cassette for plant expression
MENLTLVSCSASSPKLLIGCNFTSSLKNPTGFSRRTPNIVLRCSKISASAQSQSPSSRPENTGEIVVVKQRSKAFASIFSSSRDQQTTSVA

SEQ ID NO: 249 - protein, 76 aa, Artificial Sequence
  Targeting signal (protein) in anchoring cassette for plant expression
MEALSHVGIGLSPFQLCRLPPATTKLRRSHNTSTTICSASKWADRLLSDFNFTSDSSSSSFATATTTATLVSPPPS

SEQ ID NO: 250 - protein, 68 aa, Artificial Sequence
  Targeting signal (protein) in anchoring cassette for plant expression
MASLMLNGAITFPKGLGSPGSNLHARSIPRPTLLSVTRTSTPRLSVATRCSSSSVSSSRPSAQPRFIQ

SEQ ID NO: 251 - protein, 60 aa, Artificial Sequence
  Targeting signal (protein) in anchoring cassette for plant expression
MSLTIPANLVLNPRSNKSLTQSVPKSSARFVCSDDKSSSSTPQSMKAFSAAVALSSILLS

SEQ ID NO: 252 - protein, 50 aa, Artificial Sequence
  Targeting signal (protein) in anchoring cassette for plant expression
MASFATIAAVQPSAAVKGLGGSSLAGAKLFIKPSRQSFKTKSTRAGAVVA

SEQ ID NO: 253 - protein, 82 aa, Artificial Sequence
  Targeting signal (protein) in anchoring cassette for plant expression
MAMALQIIASSSSSPTITKSHLFSYPPLQSRYKASKPNLSSWFSLLGSSRFSPYIGLKHLGISISPKSSNPEKKRRCKSMMI

SEQ ID NO: 254 - protein, 59 aa, Artificial Sequence
  Targeting signal (protein) in anchoring cassette for plant expression
MATSVATLSSPPPVSLPLLSSSRSSFFSNCFTVTTRPNTRSLVAIGRRIRQEPTRKPLT

SEQ ID NO: 255 - protein, 38 aa, Artificial Sequence
  Targeting signal (protein) in anchoring cassette for plant expression
MSLTAQFSPPVTGITRSLRDTKPSLSNLRVFPVYTEIR

SEQ ID NO: 256 - protein, 68 aa, Artificial Sequence
  Targeting signal (protein) in anchoring cassette for plant expression
MAIRVTFTYSSYVARSIASSAGTRVGTGDVRSCFETWVRPRFCGHNQIPDIVDKSPGSNTWGPSSGPR

SEQ ID NO: 257 - protein, 45 aa, Artificial Sequence
  Targeting signal (protein) in anchoring cassette for plant expression
MATISPGGAYIGTPSPFLGKKLKPFSLTSPILSFKPTVKLNSSCR

SEQ ID NO: 258 - protein, 74 aa, Artificial Sequence
  Targeting signal (protein) in anchoring cassette for plant expression
MATAAAPAVISWTRSGIVSKSGQTQKKSEMKVSYITGLNSYGGLKAQNNKVVSMGSPLCTEQCFANVVMSLKGR

SEQ ID NO: 259 - protein, 50 aa, Artificial Sequence
  Targeting signal (protein) in anchoring cassette for plant expression
MAMSSGLFVKPTKMNHQMVRKEKIGLRISCQASSIPADRVPDMEKRKTLN

SEQ ID NO: 260 - protein, 81 aa, Artificial Sequence
  Targeting signal (protein) in anchoring cassette for plant expression
MSVSLSAASHLLCSSTRVSLSPAVTSSSSSPVVALSSSTSPHSLGSVASSSLFPHSSFVLQKKHPINGTSTRMISPKCAAS

SEQ ID NO: 261 - protein, 53 aa, Artificial Sequence
  Targeting signal (protein) in anchoring cassette for plant expression
MASLSTITQPSLVHIPGESVLHHVPSTCSFPWKPTINTKRIICSPARNSSEVS

SEQ ID NO: 262 - protein, 37 aa, Artificial Sequence
  Targeting signal (protein) in anchoring cassette for plant expression
MAASSACLLGNGLSVYTTKQRFQKLGLDRTSKVTVVK

SEQ ID NO: 263 - protein, 67 aa, Artificial Sequence
  Targeting signal (protein) in anchoring cassette for plant expression
MMVMISLHFSTPPLAFLKSDSNSRFLKNPNPNFIQFTPKSQLLFPQRLNFNTGTNLNRRTLSCYGIK

SEQ ID NO: 264 - protein, 35 aa, Artificial Sequence
  Targeting signal (protein) in anchoring cassette for plant expression
MASTSAMSLVTPLNQTRSSPFLKPLPLKPSKALVA

SEQ ID NO: 265 - protein, 62 aa, Artificial Sequence
  Targeting signal (protein) in anchoring cassette for plant expression
MAQTMLLTSGVTAGHFLRNKSPLAQPKVHHLFLSGNSPVALPSRRQSFVPLALFKPKTKAAP

SEQ ID NO: 266 - protein, 42 aa, Artificial Sequence
  Targeting signal (protein) in anchoring cassette for plant expression
MATSAIQHSSFAGQTTLKPSNDLLRKIGASNGGGRIIMRRTV

SEQ ID NO: 267 - protein, 28 aa, Artificial Sequence
  Targeting signal (protein) in anchoring cassette for plant expression
MTAILERRESESLWGRFCNWITSTENRL

SEQ ID NO: 268 - protein, 35 aa, Artificial Sequence
  Targeting signal (protein) in anchoring cassette for plant expression
MGVTKKPDLNDPVLRAKLAKGMGHNYYGEPAWPND

SEQ ID NO: 269 - protein, 61 aa, Artificial Sequence
  Targeting signal (protein) in anchoring cassette for plant expression
MGEEKSLLQFRSFPSLKTSDFALTEEPSWRLENNVSSNRRRGNKRSGGVFTNFASLSVAIR

SEQ ID NO: 270 - protein, 52 aa, Artificial Sequence
  Targeting signal (protein) in anchoring cassette for plant expression
MAIRITFTYSTHVARNLVGTRVGPGGYCFESLVRPRFFSHKRDFDRSPRNRP

SEQ ID NO: 271 - protein, 70 aa, Artificial Sequence
  Targeting signal (protein) in anchoring cassette for plant expression
MASLLSPATPTATSAAFHSCSTAGFSTPTHISSQNSSLSLLSRRGCMMRCSFSPQDIPVDSLSHLPPFLD

SEQ ID NO: 272 - protein, 57 aa, Artificial Sequence
  Targeting signal (protein) in anchoring cassette for plant expression
MASLLLPPTPTATSSTVKHNKIQTKLKPNSNAHAATCNFFLSSNSNANHRFNSAKCH

SEQ ID NO: 273 - protein, 26 aa, Artificial Sequence
  Targeting signal (protein) in anchoring cassette for plant expression
MATSARRAYGFGRADEATHPDSIRAT

SEQ ID NO: 274 - protein, 55 aa, Artificial Sequence
  Targeting signal (protein) in anchoring cassette for plant expression
MKALTLALFLALSLYLLPNPAHSRFNPIRLPTTHEPASSETPVLDINGDEVRAGG

SEQ ID NO: 275 - protein, 39 aa, Artificial Sequence
  Targeting signal (protein) in anchoring cassette for plant expression
MKALTLALFLALSLYLLPNPASSETPVLDINGDEVRAGG

SEQ ID NO: 276 - protein, 24 aa, Artificial Sequence
  Targeting signal (protein) in anchoring cassette for plant expression
MKQLLCYLPWLLLLTLLVSPLNDA

SEQ ID NO: 277 - protein, 26 aa, Artificial Sequence
  Targeting signal (protein) in anchoring cassette for plant expression
MGLVNGRASLTFLLAALTIIAMVVEA

SEQ ID NO: 278 - protein, 22 aa, Artificial Sequence
  Targeting signal (protein) in anchoring cassette for plant expression
MKCWRLSAILFLGFMLTSLSTA

SEQ ID NO: 279 - protein, 19 aa, Artificial Sequence
  Targeting signal (protein) in anchoring cassette for plant expression
MLYLVRIVTVIGVDSKKMK

SEQ ID NO: 280 - protein, 72 aa, Artificial Sequence
  Targeting signal (protein) in anchoring cassette for plant expression
MGEMEIEEIEAVLEKIWDLHDKLSDEIHLISKSHFLKSVKPSNRSEKRKNPHGNSGEDKRPGYVFIKGFAVD

SEQ ID NO: 281 - protein, 32 aa, Artificial Sequence
  Targeting signal (protein) in anchoring cassette for plant expression
MGEMEIEEIEAVLEKIWDLHDKLSDEIHLISK

SEQ ID NO: 282 - protein, 69 aa, Artificial Sequence
  Targeting signal (protein) in anchoring cassette for plant expression
MEDEEGIGLILARATELRLKISDCIDNSSTTVSDNGDGNEDLSPGEGRKSEIIGNQDKDFDSISSEDVD

SEQ ID NO: 283 - protein, 48 aa, Artificial Sequence
  Targeting signal (protein) in anchoring cassette for plant expression
MAPKKIGAGKGDSSILAKISNYDIVSQGRRAACDAVYVSKKLLKSTGK

SEQ ID NO: 284 - protein, 25 aa, Artificial Sequence
  Targeting signal (protein) in anchoring cassette for plant expression
MAAKRIGAGKSGGGDPNILARISNS

SEQ ID NO: 285 - protein, 51 aa, Artificial Sequence
  Targeting signal (protein) in anchoring cassette for plant expression
MAAKRIGAGKSGGGDPNILARISNSEIVSQGRRAAGDAVEVSKKLLRSTGK

SEQ ID NO: 286 - protein, 25 aa, Artificial Sequence
  Targeting signal (protein) in anchoring cassette for plant expression
MAAKRIGAGKSGGGDPNILARISNS

SEQ ID NO: 287 - DNA, 108 bp, Artificial Sequence; identical to SEQ ID NO: 166
  MeSH DNA in anchoring cassette for plant expression

SEQ ID NO: 288 - protein, 36 aa, Artificial Sequence; identical to SEQ ID NO: 83
  MeSH (protein) in anchoring cassette for plant expression

SEQ ID NO: 289 - protein, 108 aa, Artificial Sequence
  MeSH (protein) in anchoring cassette for plant expression
CTGAAATTGTTGAATGTGGGAGTTGGCTTACCTGTATTATTAGGAGGTGGGAAAGCGTATGCTGATGAACAAGGACCTTCCTCCTCCAGAATGTCTTATTCCAGATTT

SEQ ID NO: 290 - DNA, 153 bp, Artificial Sequence
  MeSH DNA in anchoring cassette for plant expression
atggagacaagaaaaggtgaatttattactgaagaaattaaacatgcaagtgtgcagatcatgtgtgctggtgttgtaattggactggtaactttggttggtttaaagtttttacctactaggaatggctcacccattcttcgtaaaatgact

(19) United States
(12) Patent Application Publication
(10) Pub. No.: US 2023/0183727 A1
(43) Pub. Date: Jun. 15, 2023

(54) METHODS OF OPTIMIZING TRANSGENE EXPRESSION IN PLANTS

(71) Applicant: BASF Agricultural Solutions Seed US LLC, Florham Park, NJ (US)

(72) Inventors: Desmond A Moore, Durham, NC (US); Andrew Debrecht, Cary, NC (US); Laura Schouten, Pittsboro, NC (US); Shane Hardin, Franklinton, NC (US); Brody DeYoung, Chapel Hill, NC (US); Jongmin Baek, Cary, NC (US); Shirong Zhang, Raleigh, NC (US); Soundarya Srirangan, Cary, NC (US); Allan R Wenck, Cary, NC (US)

(73) Assignee: BASF Agricultural Solutions Seed US LLC, Florham Park, NJ (US)

(21) Appl. No.: 17/768,582

(22) PCT Filed: Oct. 15, 2020
(86) PCT No.: PCT/US2020/055793

§ 371 Date: Apr. 13, 2022

Related U.S. Application Data
(60) Provisional application No. 62/916,613, filed on Oct. 17, 2019.

(51) Int. Cl. C12N 15/82; C12Q 1/6895

(52) U.S. Cl. CPC C12N 15/8221; C12N 15/8205; C12Q 1/6895; C12N 15/8286; C12Q 2600/13; C12Q 2600/158

(57) ABSTRACT

Compositions and methods for optimally expressing transgenes in organisms such as bacteria, plants, plant cells, tissues and seeds are provided. Compositions comprising the cassettes and methods of expressing are provided. The DNA constructs or expression cassettes of the invention can be used for transformation and expression in plants and bacteria wherein conventional gene expression methods result in decreased expression, negative phenotypes or reduced plant performance.

### FIELD OF THE INVENTION

This invention relates to the field of molecular biology. Provided are novel methods and expression cassettes that anchor plant defense or agronomically beneficial proteins in membranes to allow for optimal expression and performance of respective proteins. These methods and expression cassettes are useful in increasing expression levels of nucleic acids and/or proteins in plants.

### BACKGROUND OF THE INVENTION

The exchange of macromolecules between cellular compartments through various membranes is a basic biological process in eukaryotic cells and is central to the regulation of what enters or leaves a cell. Anchoring expressed transgenic proteins in various eukaryotic cellular organelle membranes can provide significant advantages over conventional nuclear transformation or organelle targeting methods for creating transgenic plants, including more abundant and reliable transgene expression, maternal inheritance, avoidance of negative phenotypes and reduction of gene or transcript silencing mechanisms.

### SUMMARY OF INVENTION

Compositions and methods for optimally expressing transgenes in plants is provided where the expressed protein is targeted and anchored into a cellular or sub-cellular organelle membrane such as for example the chloroplast outer membrane, chloroplast inner membrane, chloroplast thylakoid membrane, mitochondrial outer membrane, mitochondrial inner membrane, peroxisome membrane, plasma membrane, nuclear membrane, pre-lytic vacuolar membrane, protein storage vacuolar membrane, endosome membrane, vesicle membrane, cell wall, ER membrane and Golgi membrane (herein collectively referred to as, “Anchoring Membrane”).

In some instances of the invention certain expressed transgenes under common cell targeting strategies (e.g. constitutive expression, chloroplast targeting, ER targeting, etc.) can exhibit any one or more negative phenotype(s) such as: gene silencing, decreased gene expression, or decreased plant performance (e.g. decreased or inefficient insecticidal activity in the case of an insecticidal transgene and/or decreased yield in case of a yield gene) when expressed in a plant (herein these types of transgenes are called, “N-transgenes”). In many instances N-transgenes cannot be expressed at effective expression levels in the plant without causing some detrimental effect to the plant's health or performance. Surprisingly, the methods and embodiments as taught herein allow for higher N-transgene expression without the observed detrimental effects to plant health or performance. Applicants have unexpectedly found in multiple cases that N-transgenes can successfully be expressed in plants where the expressed protein is targeted to and anchored in an Anchoring Membrane. In some embodiments the compositions include a N-transgene that encodes for plant pesticidal, fungicidal and/or insecticidal polypeptides. Taught herein are vectors and/or plant expression cassettes comprising those N-transgenes wherein the protein encoded by the N-transgene is targeted and anchored to an Anchor Membrane by way of operably linked membrane-specific hydrophobic “MeSH targeting sequences” or interchangeably “MeSH Sequences” wherein MeSH targeting sequences are protein sequences that target a protein encoded by a N-transgene (“N-protein”) to an Anchor Membrane wherein the N-protein is fixed or anchored in the membrane wherein in some instances the membrane transects the N-protein (i.e. one portion of N-protein is located on the inner side of membrane and the other portion on the outer side of membrane). Examples of nucleotide sequences encoding MeSH Sequences can be found in SEQ ID Nos: 84-87 and 287 with respective peptide sequences depicted in SEQ ID Nos: 1-83 and 288. Generally, these nucleotide sequences encoding MeSH sequences are incorporated into plant expression cassettes along with a nucleotide encoded targeting peptide (“Targeting Sequence”) that targets the expressed protein to the respective organelle, Examples of combinations of MeSH sequences and Target Sequences can be found in Table 2. These nucleotide sequences encoding MeSH sequences and Targeting Sequences will be operably linked to a N-transgene and driven by a promoter sequence as further detailed herein and in using common techniques and know-how in the art. Embodiments also include scenarios wherein N-transgenes are genes involved in plant metabolism, genes involved in agronomic traits or abiotic traits such as yield or drought, disease resistance genes, or genes involved in plant growth or maturity. In another embodiment, the N-transgene is operably linked to a plant promoter and/or nucleotide sequences encoding targeting peptide signal and MeSH sequence. Provided are vectors that comprise a N-transgene operably linked to a promoter and nucleotide encoding MeSH sequences, herein interchageably “Anchor Cassette” or “Anchoring Cassette”. The Anchor Cassettes provided herein (see for example Table 2) can be used in the transformation and expression in organisms, including microorganisms and plants. The nucleotide or amino acid sequences associated with the Anchor Cassette may be synthetic sequences that have been designed for expression in an organism including, but not limited to, a microorganism or a plant. Compositions also comprise bacteria, plants, plant cells, tissues, and seeds comprising and/or expressing the Anchor Cassettes of the invention.

In one embodiment, Anchor Cassettes are provided wherein a N-transgene encodes a pesticidal, fungicidal or insecticidal protein that kills or inhibits crop pest (herein, “pesticidal N-transgene”) wherein said pesticidal N-Transgene is in an Anchor Cassette and said Anchor cassette expresses a pesticidal N-Protein comprising one or more MeSH sequences that targets said pesticidal N-Protein to an Anchor Membrane that confers increased expression and/or performance as compared to a control cassette comprising only the N-Transgene and no membrane anchoring mechanism (i.e. MeSH sequences).

In one embodiment, the MeSH sequences are encoded by nucleotide sequences comprising any one of the following and/or homologs having at least 70, 75, 80, 85, 90, 95, 97, 98, 99 or 100% sequence similarity to: Atstn8 (SEQ ID NO: 84), Gmftsh9 I (SEQ ID NO: 85); Gmftsh9 I & II (SEQ ID NO: 86), GmArc6 (SEQ ID NO: 87); Stn8+(SEQ ID NO: 287) and/or any of the respective MeSH sequences as depicted in SEQ ID Nos: 88-177 (herein, “Nucleotide MeSH Sequences”).

In one embodiment, an Anchor Cassette is provided comprising a promoter operably linked to a N-transgene and one or more Nucleotide MeSH Sequences. The promoter can be a constitutive promoter or can be a tissue specific promoter. Promoters used in the invention can either be constitutive, tissue specific or inducible promoters. In another embodiment, the Anchor Cassette comprises a promoter operably linked to a N-transgene, one or more nucleotide sequences encoding MeSH sequences and a targeting peptide. Examples of possible plant Anchor Cassettes are depicted in Table 2.

In another embodiment the plant can either be a monocot or dicot plant, In further embodiments the plant can be any one of the following: corn (maize), sorghum, wheat, sunflower, tomato, crucifers, peppers, potato, cotton, rice, soybean, sugar beet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

### DETAILED DESCRIPTION

The present invention is drawn to compositions and methods of expressing N-transgenes in organisms, particularly plants or plant cells. The term “increased N-transgene performance” or interchangeably “increased performance” means the N-transgene has improved performance as compared to a control plant in relation to relative N-transgene expression and/or gene activity and may further exhibit a decreased incidence of any of the following: negative plant phenotype, reduced plant performance or plant health, reduced gene expression or any other factors associated with the respective N-transgene when expressed in an alternative fashion of not anchoring the expressed N-Protein into a respective membrane.

In several embodiments the N-transgene is a gene that confers in a plant increased insect resistance and/or insect tolerance. Some examples of N-transgenes may be found in the Examples herein. Herein, “resistance” is intended that the pest (e.g., insect) is killed upon ingestion or other contact with the polypeptides of the invention. By “tolerance” is intended an impairment or reduction in the movement, feeding, reproduction, or other functions of the pest. Various embodiments herein involve transforming an Anchor Cassette comprising N-transgenes encoding for pesticidal proteins that either exhibit increased plant resistance or plant tolerance to the target pest as compared to a control plant. In particular, the nucleotide sequences of the invention are useful for preparing plants and microorganisms that possess pesticidal activity. Thus, transformed bacteria, plants, plant cells, plant tissues and seeds are provided comprising the Anchor Cassettes as described herein. The sequences find use in the construction of expression vectors comprising Anchor Cassettes for subsequent transformation into organisms of interest (see for example, Table 2). The N-transgene, in some embodiments, encode proteins having pesticidal activity against lepidopteran, hemipteran, coleopteran, dipteran, and nematode pest populations or in some instances the N-transgene may be a gene that encodes proteins that are active against fungi (i.e. fungicidal proteins). The term “pesticidal toxin” or “pesticidal protein” refers to a toxin that has toxic activity against one or more pests, including, but not limited to, members of the Lepidoptera, Diptera, Hemiptera, and Coleoptera orders, or the Nematoda phylum, or a protein that has a high degree (>80% sequence identity) of homology to such a protein.

An Anchoring Cassette of the invention may be provided in an expression cassette for expression in a host cell of interest, e.g. a plant cell or a microbe (e.g. Agrobacterium tumefaciens or E. Coli). By “plant expression cassette” is intended an Anchoring Cassette that is capable of resulting in the expression of a N-transgene protein, MeSH sequence and optionally a targeting peptide from an open reading frame in a plant cell.

By “signal sequence” is intended a sequence that is known or suspected to result in co-translational or post-translational peptide transport across the cell membrane. In eukaryotes, this typically involves secretion into the Golgi apparatus, with some resulting glycosylation. Insecticidal toxins of bacteria are often synthesized as protoxins, which are protolytically activated in the gut of the target pest (Chang (1987) Methods Enzymol. 153:507-516). In some embodiments of the present invention, the signal sequence is located in the native sequence, or may be derived from a sequence of the invention. By “leader sequence” is intended any sequence that when translated, results in an amino acid sequence sufficient to trigger co-translational transport of the peptide chain to a subcellular organelle. Thus, this includes leader sequences targeting transport and/or glycosylation by passage into the endoplasmic reticulum, passage to vacuoles, plastids including chloroplasts, mitochondria, and the like. Thus, further provided herein is a polypeptide comprising an amino acid sequence of the present invention that is operably linked to a heterologous leader or signal sequence.

By “plant transformation vector” is intended a DNA molecule that is necessary for efficient transformation of a plant cell. Such a molecule may consist of one or more plant expression cassettes, and may be organized into more than one “vector” DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a nucleic acid construct designed for transfer between different host cells. “Expression vector” refers to a vector that has the ability to incorporate, integrate and express heterologous DNA sequences or fragments in a foreign cell. The cassette will include 5′ and/or 3′ regulatory sequences operably linked to a sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. In some embodiments, the nucleotide sequence is operably linked to a heterologous promoter capable of directing expression of said nucleotide sequence in a host cell, such as a microbial host cell or a plant host cell. The cassette may additionally contain at least one additional gene to be co-transformed into the organism. Alternatively, the additional gene(s) can be provided on multiple expression cassettes.

In various embodiments, the nucleotide sequence of the invention is operably linked to a heterologous promoter capable of directing expression of the nucleotide sequence in a cell, e.g., in a plant cell or a microbe. “Promoter” refers to a nucleic acid sequence that functions to direct transcription of a downstream coding sequence. The promoter together with other transcriptional and translational regulatory nucleic acid sequences (also termed “control sequences”) are necessary for the expression of a DNA sequence of interest.

Such an expression cassette is provided with a plurality of restriction sites for insertion of the pesticidal sequence to be under the transcriptional regulation of the regulatory regions.

The expression cassette will include in the 5′-3′ direction of transcription, a transcriptional and translational initiation region (i.e., a promoter), a DNA sequence of the invention, and a translational and transcriptional termination region (i.e., termination region) functional in plants. The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds (1987) Nucleic Acids Res. 15:2343-2361. Also, the location of the promoter relative to the transcription start may be optimized. See, e.g., Roberts et al (1979) Proc. Natl. Acad. Sci. USA, 76:760-764. Many suitable promoters for use in plants are well known in the art.

For instance, suitable constitutive promoters for use in plants include: the promoters from plant viruses, such as the peanut chlorotic streak caulimovirus (PC1SV) promoter (U.S. Pat. No. 5,850,019); the 35S promoter from cauliflower mosaic virus (CaMV) (Odell et al. (1985) Nature 313:810-812); the 35S promoter described in Kay et al. (1987) Science 236: 1299-1302; promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171 and U.S. Pat. No. 5,641,876); ubiquitin (Christensen et al. (1989) Plant Mol. Biol. 12:619-632 and Christensen et al. (1992) Plant Mol. Biol. 18:675-689) and Grefen et al. (2010) Plant J, 64:355-365; pEMU (Last et al. (1991) Theor. Appl. Genet. 81:581-588); MAS (Velten et al. (1984) EMBO J. 3:2723-2730 and U.S. Pat. No. 5,510,474); maize H3 histone (Lepetit et al. (1992) Mol. Gen. Genet. 231:276-285 and Atanassova et al. (1992) Plant J. 2(3):291-300); Brassica napus ALS3 (PCT application WO97/41228); a plant ribulose-biscarboxylase/oxygenase (RuBisCO) small subunit gene; the circovirus (AU 689 311) or the Cassava vein mosaic virus (CsVMV, U.S. Pat. No. 7,053,205); promoters from soybean (Pbdc6 or Pbdc7, described in WO/2014/150449 or ubiquitin 3 promoter described in U.S. Pat. Nos. 7,393,948 and 8,395,021); and promoters of various Agrobacterium genes (see U.S. Pat. Nos. 4,771,002; 5,102,796; 5,182,200; and 5,428,147).

In certain preferred embodiments of the invention, leaf preferred or leaf specific promoters would be ideal in the case of the expression of insecticidal/pesticidal N-transgenes and creating plants resistant to or tolerant to phytophagous insects or other plant pests.

Suitable inducible promoters for use in plants include: the promoter from the ACE1 system which responds to copper (Mett et al. (1993) PNAS 90:4567-4571); the promoter of the maize In2 gene which responds to benzenesulfonamide herbicide safeners (Hershey et al. (1991) Mol. Gen. Genetics 227:229-237 and Gatz et al. (1994) Mol. Gen. Genetics 243:32-38); and the promoter of the Tet repressor from Tn10 (Gatz et al. (1991) Mol. Gen. Genet. 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al. (1991) Proc. Natl. Acad. Sci. USA 88:10421) or the recent application of a chimeric transcription activator, XVE, for use in an estrogen receptor-based inducible plant expression system activated by estradiol (Zuo et al. (2000) Plant 1, 24:265-273). Other inducible promoters for use in plants are described in EP 332104, PCT WO 93/21334 and PCT WO 97/06269 which are herein incorporated by reference in their entirety. Promoters composed of portions of other promoters and partially or totally synthetic promoters can also be used. See, e.g., Ni et al. (1995) Plant J. 7:661-676 and PCT WO 95/14098 describing such promoters for use in plants.

In one embodiment of this invention, a promoter sequence specific for particular regions or tissues of plants can be used to express the N-transgenes of the invention, such as promoters specific for seeds (Datla, R. et al., 1997, Biotechnology Ann. Rev. 3, 269-296), especially the napin promoter (EP 255 378 A1), the phaseolin promoter, the glutenin promoter, the helianthinin promoter (WO92/17580), the albumin promoter (WO98/45460), the oleosin promoter (WO98/45461), the SAT1 promoter or the SAT3 promoter (PCT/US98/06978).

Use may also be made of an inducible promoter advantageously chosen from the phenylalanine ammonia lyase (PAL), HMG-CoA reductase (HMG), chitinase, glucanase, proteinase inhibitor (PI), PR1 family gene, nopaline synthase (nos) and vspB promoters (U.S. Pat. No. 5,670,349, Table 3), the HMG2 promoter (U.S. Pat. No. 5,670,349), the apple beta-galactosidase (ABG1) promoter and the apple aminocyclopropane carboxylate synthase (ACC synthase) promoter (WO98/45445). Multiple promoters can be used in the constructs of the invention, including in succession.

The promoter may include, or be modified to include, one or more enhancer elements. In some embodiments, the promoter may include a plurality of enhancer elements. Promoters containing enhancer elements provide for higher levels of transcription as compared to promoters that do not include them. Suitable enhancer elements for use in plants include the PC1SV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156); the translation activator of the tobacco mosaic virus (TMV) described in Application WO87/07644, or of the tobacco etch virus (TEV) described by Carrington & Freed 1990, J. Virol. 64: 1590-1597, for example, or introns such as the adh1 intron of maize or intron 1 of rice actin. See also PCT WO96/23898, WO2012/021794, WO2012/021797, WO2011/084370, and WO2011/028914.

Often, such constructs can contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. By “signal sequence” is intended a sequence that is known or suspected to result in co-translational or post-translational peptide transport across the cell membrane. In eukaryotes, this typically involves secretion into the Golgi apparatus, with some resulting glycosylation. By “leader sequence” is intended any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. It may also be preferable to engineer the plant expression cassette to contain an intron, such that mRNA processing of the intron is required for expression.

By “3′ untranslated region” is intended a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a polynucleotide located upstream of a coding sequence.

Other upstream or downstream untranslated elements include enhancers. Enhancers are polynucleotides that act to increase the expression of a promoter region. Enhancers are well known in the art and include, but are not limited to, the SV40 enhancer region and the 35S enhancer element.

The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639. Where appropriate, the gene(s) may be optimized for increased expression in the transformed host cell (synthetic DNA sequence). That is, the genes can be synthesized using host cell-preferred codons for improved expression, or may be synthesized using codons at a host-preferred codon usage frequency. Expression of the open reading frame of the synthetic DNA sequence in a cell results in production of the polypeptide of the invention. Synthetic DNA sequences can be useful to simply remove unwanted restriction endonuclease sites, to facilitate DNA cloning strategies, to alter or remove any potential codon bias, to alter or improve GC content, to remove or alter alternate reading frames, and/or to alter or remove intron/exon splice recognition sites, polyadenylation sites, Shine-Delgarno sequences, unwanted promoter elements and the like that may be present in a native DNA sequence. Generally, the GC content of the gene will be increased. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, U.S. Patent Publication No. 20090137409, and Murray et al. (1989) Nucleic Acids Res. 17:477-498, herein incorporated by reference.

It is also possible that synthetic DNA sequences may be utilized to introduce other improvements to a DNA sequence, such as introduction of an intron sequence, creation of a DNA sequence that is expressed as a protein fusion to organelle targeting sequences, such as chloroplast transit peptides, apoplast/vacuolar targeting peptides, or peptide sequences that result in retention of the resulting peptide in the endoplasmic reticulum. Thus, in one embodiment, the pesticidal protein is targeted to the chloroplast for expression. In this manner, where the pesticidal protein is not directly inserted into the chloroplast, the expression cassette will additionally contain a nucleic acid encoding a transit peptide to direct the pesticidal protein to the chloroplasts. Such transit peptides are known in the art. See, for example, Von Heijne et al. (1991) Plant Mol. Biol. Rep. 9:104-126; Clark et al. (1989)J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The N-Protein to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

### Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods.

By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

The transgenic plants of the invention express one or more of the novel N-transgene sequences disclosed herein wherein the expressed protein is preferably targeted to and anchored in an Anchoring Membrane. In some embodiments, the protein or nucleotide sequence of the invention is advantageously combined in plants with other genes which encode proteins or RNAs that confer useful agronomic properties to such plants. Among the genes which encode proteins or RNAs that confer useful agronomic properties on the transformed plants, mention can be made of the DNA sequences encoding proteins which confer tolerance to one or more herbicides, and others which confer tolerance to certain insects, those which confer tolerance to certain diseases, DNAs that encodes RNAs that provide nematode or insect control, and the like. Such genes are in particular described in published PCT Patent Applications WO91/02071 and WO95/06128 and in U.S. Pat. No. 7,923,602 and US Patent Application Publication No. 20100166723, each of which is herein incorporated by reference in its entirety.

Among the DNA sequences encoding proteins which confer tolerance to certain herbicides on the transformed plant cells and plants, mention can be made of a bar or PAT gene or the Streptomyces coelicolor gene described in WO2009/152359 which confers tolerance to glufosinate herbicides, a gene encoding a suitable EPSPS which confers tolerance to herbicides having EPSPS as a target, such as glyphosate and its salts (U.S. Pat. Nos. 4,535,060, 4,769,061, 5,094,945, 4,940,835, 5,188,642, 4,971,908, 5,145,783, 5,310,667, 5,312,910, 5,627,061, 5,633,435), a gene encoding glyphosate-n-acetyltransferase (for example, U.S. Pat. Nos. 8,222,489, 8,088,972, 8,044,261, 8,021,857, 8,008,547, 7,999,152, 7,998,703, 7,863,503, 7,714,188, 7,709,702, 7,666,644, 7,666,643, 7,531,339, 7,527,955, and 7,405,074), a gene encoding glyphosate oxydoreductase (for example, U.S. Pat. No. 5,463,175), or a gene encoding an HPPD inhibitor-tolerant protein (for example, the HPPD inhibitor tolerance genes described in WO 2004/055191, WO 199638567, U.S. Pat. No. 6,791,014, WO2011/068567, WO2011/076345, WO2011/085221, WO2011/094205, WO2011/068567, WO2011/094199, WO2011/094205, WO2011/145015, WO2012/056401, and WO2014/043435).

Among the DNA sequences encoding a suitable EPSPS which confer tolerance to the herbicides which have EPSPS as a target, mention will more particularly be made of the gene which encodes a plant EPSPS, in particular maize EPSPS, particularly a maize EPSPS which comprises two mutations, particularly a mutation at amino acid position 102 and a mutation at amino acid position 106 (WO2004/074443), and which is described in Patent Application U.S. Pat. No. 6,566,587, hereinafter named double mutant maize EPSPS or 2mEPSPS, or the gene which encodes an EPSPS isolated from Agrobacterium and which is described by sequence ID No. 2 and sequence ID No. 3 of U.S. Pat. No. 5,633,435, also named CP4.

Among the DNA sequences encoding a suitable EPSPS which confer tolerance to the herbicides which have EPSPS as a target, mention will more particularly be made of the gene which encodes an EPSPS GRG23 from Arthrobacter globiformis, but also the mutants GRG23 ACE1, GRG23 ACE2, or GRG23 ACE3, particularly the mutants or variants of GRG23 as described in WO2008/100353, such as GRG23(ace3)R173K of SEQ ID No. 29 in WO2008/100353.

In the case of the DNA sequences encoding EPSPS, and more particularly encoding the above genes, the sequence encoding these enzymes is advantageously preceded by a sequence encoding a transit peptide, in particular the “optimized transit peptide” described in U.S. Pat. No. 5,510,471 or 5,633,448.

Exemplary herbicide tolerance traits that can be combined with the nucleic acid sequence of the invention further include at least one ALS (acetolactate synthase) inhibitor (WO2007/024782); a mutated Arabidopsis ALS/AHAS gene (U.S. Pat. No. 6,855,533); genes encoding 2,4-D-monooxygenases conferring tolerance to 2,4-D (2,4-dichlorophenoxyacetic acid) by metabolization (U.S. Pat. No. 6,153,401); and, genes encoding Dicamba monooxygenases conferring tolerance to dicamba (3,6-dichloro-2-methoxybenzoic acid) by metabolization (US 2008/0119361 and US 2008/0120739).

In various embodiments, the nucleic acid of the invention is stacked with one or more herbicide tolerant genes, including one or more HPPD inhibitor herbicide tolerant genes, and/or one or more genes tolerant to glyphosate and/or glufosinate.

Among the DNA sequences encoding proteins concerning properties of tolerance to insects, mention will more particularly be made of the Bt proteins widely described in the literature and well known to those skilled in the art. Mention will also be made of proteins extracted from bacteria such as Photorhabdus (WO97/17432 & WO98/08932).

Among such DNA sequences encoding proteins of interest which confer novel properties of tolerance to insects, mention will more particularly be made of the Bt Cry or VIP proteins widely described in the literature and well known to those skilled in the art. These include the Cry1F protein or hybrids derived from a Cry 1F protein (e.g., the hybrid Cry1A-Cry1F proteins described in U.S. Pat. Nos. 6,326,169; 6,281,016; 6,218,188, or toxic fragments thereof), the Cry1A-type proteins or toxic fragments thereof, preferably the Cry1Ac protein or hybrids derived from the Cry1Ac protein (e.g., the hybrid Cry1Ab-Cry1Ac protein described in U.S. Pat. No. 5,880,275) or the Cry1Ab or Bt2 protein or insecticidal fragments thereof as described in EP451878, the Cry2Ae, Cry2Af or Cry2Ag proteins as described in WO2002/057664 or toxic fragments thereof, the Cry1A.105 protein described in WO 2007/140256 (SEQ ID No. 7) or a toxic fragment thereof, the VIP3Aa19 protein of NCBI accession ABG20428, the VIP3Aa20 protein of NCBI accession ABG20429 (SEQ ID No. 2 in WO 2007/142840), the VIP3A proteins produced in the COT202 or COT203 cotton events (WO2005/054479 and WO2005/054480, respectively), the Cry proteins as described in WO2001/47952, the VIP3Aa protein or a toxic fragment thereof as described in Estruch et al. (1996), Proc Natl Acad Sci USA. 28;93(11):5389-94 and U.S. Pat. No. 6,291,156, the insecticidal proteins from Xenorhabdus (as described in WO98/50427), Serratia (particularly from S. entomophila) or Photorhabdus species strains, such as Tc-proteins from Photorhabdus as described in WO98/08932 (e.g., Waterfield et al., 2001, Appl Environ Microbiol. 67(11):5017-24; Ffrench-Constant and Bowen, 2000, Cell Mol Life Sci.; 57(5):828-33). Also any variants or mutants of any one of these proteins differing in some (1-10, preferably 1-5) amino acids from any of the above sequences, particularly the sequence of their toxic fragment, or which are fused to a transit peptide, such as a plastid transit peptide, or another protein or peptide, is included herein.

In various embodiments, the nucleic acid of the invention can be combined in plants with one or more genes conferring a desirable trait, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like.

Particularly useful transgenic events which may be combined with the genes of the current invention in plants of the same species (e.g., by crossing or by re-transforming a plant containing another transgenic event with a chimeric gene of the invention), include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control-herbicide tolerance, deposited as PTA-6233, described in WO2005/103266 or US-A 2005-216969); Event 3006-210-23 (cotton, insect control-herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control-herbicide tolerance, deposited as ATCC PTA-11508, described in WO 11/075593); Event 43A47 (corn, insect control-herbicide tolerance, deposited as ATCC PTA-11509, described in WO2011/075595); Event 5307 (corn, insect control, deposited as ATCC PTA-9561, described in WO2010/077816), Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480);); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control-herbicide tolerance, deposited as ATCC PTA 11384, described in US-A 2006-070139); Event DAS-59132 (corn, insect control-herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-1 (brinjal, insect control, not deposited, described in WO 07/091277); Event FI117 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control-herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140), Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control-herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MS11 (oilseed rape, pollination control-herbicide tolerance, deposited as ATCC PTA-850 or PTA-2485, described in WO2001/031042); Event MS8 (oilseed rape, pollination control-herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control-herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control-herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control-herbicide tolerance, not deposited, described in US-A 2005-039226 or WO2004/099447); Event VIP1034 (corn, insect control-herbicide tolerance, deposited as ATCC PTA-3925., described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession N° PTA-11041) optionally stacked with event EE-GM1/LL27 or event EE-GM2/LL55 (WO2011/063413A2), event DAS-68416-4 (soybean, herbicide tolerance, ATCC Accession N° PTA-10442, WO2011/066360A1), event DAS-68416-4 (soybean, herbicide tolerance, ATCC Accession N° PTA-10442, WO2011/066384A1), event DP-040416-8 (corn, insect control, ATCC Accession N° PTA-11508, WO2011/075593A1), event DP-043A47-3 (corn, insect control, ATCC Accession N° PTA-11509, WO2011/075595A1), event DP-004114-3 (corn, insect control, ATCC Accession N° PTA-11506, WO2011/084621A1), event DP-032316-8 (corn, insect control, ATCC Accession N° PTA-11507, WO2011/084632A1), event MON-88302-9 (oilseed rape, herbicide tolerance, ATCC Accession N° PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession N°. PTA-10296, WO2012/051199A2), event DAS-44406-6 (soybean, stacked herbicide tolerance, ATCC Accession N°. PTA-11336, WO2012/075426A1), event DAS-14536-7 (soybean, stacked herbicide tolerance, ATCC Accession N°. PTA-11335, WO2012/075429A1), event SYN-000H2-5 (soybean, herbicide tolerance, ATCC Accession N°. PTA-11226, WO2012/082548A2), event DP-061061-7 (oilseed rape, herbicide tolerance, no deposit N° available, WO2012071039A1), event DP-073496-4 (oilseed rape, herbicide tolerance, no deposit N° available, US2012131692), event 8264.44.06.1 (soybean, stacked herbicide tolerance, Accession N° PTA-11336, WO2012075426A2), event 8291.45.36.2 (soybean, stacked herbicide tolerance, Accession N°. PTA-11335, WO2012075429A2), event SYHTOH2 (soybean, ATCC Accession N°. PTA-11226, WO2012/082548A2), event MON88701 (cotton, ATCC Accession N° PTA-11754, WO2012/134808A1), event KK179-2 (alfalfa, ATCC Accession N° PTA-11833, WO2013/003558A1), event pDAB8264.42.32.1 (soybean, stacked herbicide tolerance, ATCC Accession N° PTA-11993, WO2013/010094A1), event MZDT09Y (corn, ATCC Accession N° PTA-13025, WO2013/012775A1).

Transformation of plant cells can be accomplished by one of several techniques known in the art. The pesticidal gene of the invention may be modified to obtain or enhance expression in plant cells. Typically, a construct that expresses such a protein would contain a promoter to drive transcription of the gene, as well as a 3′ untranslated region to allow transcription termination and polyadenylation. The organization of such constructs is well known in the art. In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression. Preferably the plant expression cassette is a Anchoring Cassette.

Typically, this “plant expression cassette” or preferably the Anchoring Cassette will be inserted into a “plant transformation vector”. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the pesticidal gene preferably the N-Transgene in form an Anchoring Cassette are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997)Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation (U.S. Published Application No. 20010026941; U.S. Pat. No. 4,945,050; International Publication No. WO 91/00915; U.S. Published Application No. 2002015066), Lec1 transformation, and various other non-particle direct-mediated methods to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette preferably an Anchoring Cassette of the invention, stably incorporated into their genome.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformant, fractionated in a formaldehyde agarose gel, and blotted onto a nylon filter according to standard procedures that are routinely used in the art (Sambrook and Russell, 2001, supra). Expression of RNA encoded by the pesticidal gene is then tested by hybridizing the filter to a radioactive probe derived from a pesticidal gene, by methods known in the art (Sambrook and Russell, 2001, supra).

Western blot, biochemical assays and the like may be carried out on the transgenic plants to confirm the presence of protein encoded by the pesticidal gene by standard procedures (Sambrook and Russell, 2001, supra) using antibodies that bind to one or more epitopes present on the pesticidal protein.

### N-Transgene Pesticidal Activity in Plants

In another aspect of the invention, one may generate transgenic plants expressing a pesticidal protein (i.e. N-Protein) that has increased expression as compared to a control plant. Methods described above by way of example may be utilized to generate transgenic plants, but the manner in which the transgenic plant cells are generated is not critical to this invention. Methods known or described in the art such as Agrobacterium-mediated transformation, biolistic transformation, and non-particle-mediated methods may be used at the discretion of the experimenter. Plants expressing a N-transgene by way of an Anchoring cassette may be isolated by common methods described in the art, for example by transformation of callus, selection of transformed callus, and regeneration of fertile plants from such transgenic callus. In such process, one may use any gene as a selectable marker so long as its expression in plant cells confers ability to identify or select for transformed cells.

A number of markers have been developed for use with plant cells, such as resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J. Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing a N-transgene protein may be tested for gene expression and activity, and the plants showing optimal activity and performance can be selected for further breeding. Methods are available in the art to assay for pest activity. Generally, the protein is mixed and used in feeding assays. See, for example Marrone et al. (1985)1 of Economic Entomology 78:290-293. Methods are also known in the art to assay for plant yield, plant disease resistance, etc.

The present invention may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape., etc.).

“Pest” includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera.

The order Coleoptera includes the suborders Adephaga and Polyphaga. Suborder Adephaga includes the superfamilies Caraboidea and Gyrinoidea, while suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Hemipteran pests (which include species that are designated as Hemiptera, Homoptera, or Heteroptera) include, but are not limited to, Lygus spp., such as Western tarnished plant bug (Lygus hesperus), the tarnished plant bug (Lygus lineolaris), and green plant bug (Lygus elisus); aphids, such as the green peach aphid (Myzus persicae), cotton aphid (Aphis gossypii), cherry aphid or black cherry aphid (Myzus cerasi), soybean aphid (Aphis glycines Matsumura); brown plant hopper (Nilaparvata lugens), and rice green leafhopper (Nephotettix spp.); and stink bugs, such as green stink bug (Acrosternum hilare), brown marmorated stink bug (Halyomorpha halys), southern green stink bug (Nezara viridula), rice stink bug (Oebalus pugnax), forest bug (Pentatoma rufipes), European stink bug (Rhaphigaster nebulosa), and the shield bug Troilus luridus.

Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Spodoptera cosmioides; Spodoptera eridania; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Siphaflava, yellow sugarcane aphid; Blissus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grand's, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differential's, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodopterafrugiperda, fall armyworm; Spodoptera cosmioides; Spodoptera eridania; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Chilu suppressalis, Asiatic rice borer; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Spodoptera cosmioides; Spodoptera eridania; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus, chinch bug; Acrosternum hilare, green stink bug; Euschistus servus, brown stink bug; Euschistus heros, neotropical brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

### Methods for Increasing Plant Yield

Methods for increasing plant yield are provided. In cases where the expression of a N-transgene results in decreased plant yield the methods and Anchoring cassettes herein provide a means to increase plant yield in these cases. The methods comprise providing a plant or plant cell expressing an Anchoring Cassette with the N-transgene of interest thus anchoring the N-transgene encoded protein into a membrane and allowing for higher gene expression and performance. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not expressing the pesticidal sequence. In specific methods, plant yield is increased as a result of improved pest resistance of a plant expressing a pesticidal protein disclosed herein. Expression of the pesticidal protein results in a reduced ability of a pest to infest or feed.

In some embodiments the N-transgene may encode a protein that confers herbicide resistance to any of the following in a plant: Fruits/Vegetables Herbicides: Atrazine, Bromacil, Diuron, Glyphosate, Linuron, Metribuzin, Simazine, Trifluralin, Fluazifop, Glufosinate, Halosulfuron Gowan, Paraquat, Propyzamide, Sethoxydim, Butafenacil, Halosulfuron, Indaziflam; Fruits/Vegetables Insecticides:

Aldicarb, Bacillus thuriengiensis, Carbaryl, Carbofuran, Chlorpyrifos, Cypermethrin, Deltamethrin, Abamectin, Cyfluthrin/beta-cyfluthrin, Esfenvalerate, Lambda-cyhalothrin, Acequinocyl, Bifenazate, Methoxyfenozide, Novaluron, Chromafenozide, Thiacloprid, Dinotefuran, Fluacrypyrim, Spirodiclofen, Gamma-cyhalothrin, Spiromesifen, Spinosad, Rynaxypyr, Cyazypyr, Triflumuron, Spirotetramat, Imidacloprid, Flubendiamide, Thiodicarb, Metaflumizone, Sulfoxaflor, Cyflumetofen, Cyanopyrafen, Clothianidin, Thiamethoxam, Spinotoram, Thiodicarb, Flonicamid, Methiocarb, Emamectin-benzoate, Indoxacarb, Fenamiphos, Pyriproxifen, Fenbutatin-oxid; Fruits/Vegetables Fungicides: Ametoctradin, Azoxystrobin, Benthiavalicarb, Boscalid, Captan, Carbendazim, Chlorothalonil, Copper, Cyazofamid, Cyflufenamid, Cymoxanil, Cyproconazole, Cyprodinil, Difenoconazole, Dimetomorph, Dithianon, Fenamidone, Fenhexamid, Fluazinam, Fludioxonil, Fluopicolide, Fluopyram, Fluoxastrobin, Fluxapyroxad, Folpet, Fosetyl, Iprodione, Iprovalicarb, Isopyrazam, Kresoxim-methyl, Mancozeb, Mandipropamid, Metalaxyl/mefenoxam, Metiram, Metrafenone, Myclobutanil, Penconazole, Penthiopyrad, Picoxystrobin, Propamocarb, Propiconazole, Propineb, Proquinazid, Prothioconazole, Pyraclostrobin, Pyrimethanil, Quinoxyfen, Spiroxamine, Sulphur, Tebuconazole, Thiophanate-methyl, Trifloxystrobin; Cereals Herbicides: 2.4-D, Amidosulfuron, Bromoxynil, Carfentrazone-E, Chlorotoluron, Chlorsulfuron, Clodinafop-P, Clopyralid, Dicamba, Diclofop-M, Diflufenican, Fenoxaprop, Florasulam, Flucarbazone-NA, Flufenacet, Flupyrosulfuron-M, Fluroxypyr, Flurtamone, Glyphosate, Iodosulfuron, Ioxynil, Isoproturon, MCPA, Mesosulfuron, Metsulfuron, Pendimethalin, Pinoxaden, Propoxycarbazone, Prosulfocarb, Pyroxsulam, Sulfosulfuron, Thifensulfuron, Tralkoxydim, Triasulfuron, Tribenuron, Trifluralin, Tritosulfuron; Cereals Fungicides: Azoxystrobin, Bixafen, Boscalid, Carbendazim, Chlorothalonil, Cyflufenamid, Cyproconazole, Cyprodinil, Dimoxystrobin, Epoxiconazole, Fenpropidin, Fenpropimorph, Fluopyram, Fluoxastrobin, Fluquinconazole, Fluxapyroxad, Isopyrazam, Kresoxim-methyl, Metconazole, Metrafenone, Penthiopyrad, Picoxystrobin, Prochloraz, Propiconazole, Proquinazid, Prothioconazole, Pyraclostrobin, Quinoxyfen, Spiroxamine, Tebuconazole, Thiophanate-methyl, Trifloxystrobin; Cereals Insecticides: Dimethoate, Lambda-cyhalthrin, Deltamethrin, alpha-Cypermethrin, l3-cyfluthrin, Bifenthrin, Imidacloprid, Clothianidin, Thiamethoxam, Thiacloprid, Acetamiprid, Dinetofuran, Clorphyriphos, Pirimicarb, Methiocarb, Sulfoxaflor; Maize Herbicides: Atrazine, Alachlor, Bromoxynil, Acetochlor, Dicamba, Clopyralid, (S-)Dimethenamid, Glufosinate, Glyphosate, Isoxaflutole, (S-)Metolachlor, Mesotrione, Nicosulfuron, Primisulfuron, Rimsulfuron, Sulcotrione, Foramsulfuron, Topramezone, Tembotrione, Saflufenacil, Thiencarbazone, Flufenacet, Pyroxasulfon; Maize Insecticides: Carbofuran, Chlorpyrifos, Bifenthrin, Fipronil, Imidacloprid, Lambda-Cyhalothrin, Tefluthrin, Terbufos, Thiamethoxam, Clothianidin, Spiromesifen, Flubendiamide, Triflumuron, Rynaxypyr, Deltamethrin, Thiodicarb, B-Cyfluthrin, Cypermethrin, Bifenthrin, Lufenuron, Tebupirimphos, Ethiprole, Cyazypyr, Thiacloprid, Acetamiprid, Dinetofuran, Avermectin; Maize Fungicides: Azoxystrobin, Bixafen, Boscalid, Cyproconazole, Dimoxystrobin, Epoxiconazole, Fenitropan, Fluopyram, Fluoxastrobin, Fluxapyroxad, Isopyrazam, Metconazole, Penthiopyrad, Picoxystrobin, Propiconazole, Prothioconazole, Pyraclostrobin, Tebuconazole, Trifloxystrobin; Rice Herbicides: Butachlor, Propanil, Azimsulfuron, Bensulfuron, Cyhalofop, Daimuron, Fentrazamide, Imazosulfuron, Mefenacet, Oxaziclomefone, Pyrazosulfuron, Pyributicarb, Quinclorac, Thiobencarb, Indanofan, Flufenacet, Fentrazamide, Halosulfuron, Oxaziclomefone, Benzobicyclon, Pyriftalid, Penoxsulam, Bispyribac, Oxadiargyl, Ethoxysulfuron, Pretilachlor, Mesotrione, Tefuryltrione, Oxadiazone, Fenoxaprop, Pyrimisulfan; Rice Insecticides: Diazinon, Fenobucarb, Benfuracarb, Buprofezin, Dinotefuran, Fipronil, Imidacloprid, Isoprocarb, Thiacloprid, Chromafenozide, Clothianidin, Ethiprole, Flubendiamide, Rynaxypyr, Deltamethrin, Acetamiprid, Thiamethoxam, Cyazypyr, Spinosad, Spinotoram, Emamectin-Benzoate, Cypermethrin, Chlorpyriphos, Etofenprox, Carbofuran, Benfuracarb, Sulfoxaflor; Rice Fungicides: Azoxystrobin, Carbendazim, Carpropamid, Diclocymet, Difenoconazole, Edifenphos, Ferimzone, Gentamycin, Hexaconazole, Hymexazol, Iprobenfos (IBP), Isoprothiolane, Isotianil, Kasugamycin, Mancozeb, Metominostrobin, Orysastrobin, Pencycuron, Probenazole, Propiconazole, Propineb, Pyroquilon, Tebuconazole, Thiophanate-methyl, Tiadinil, Tricyclazole, Trifloxystrobin, Validamycin; Cotton Herbicides: Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron; Cotton Insecticides: Acephate, Aldicarb, Chlorpyrifos, Cypermethrin, Deltamethrin, Abamectin, Acetamiprid, Emamectin Benzoate, Imidacloprid, Indoxacarb, Lambda-Cyhalothrin, Spinosad, Thiodicarb, Gamma-Cyhalothrin, Spiromesifen, Pyridalyl, Flonicamid Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor; Cotton Fungicides: Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin; Soybean Herbicides: Alachlor, Bentazone, Trifluralin, Chlorimuron-Ethyl, Cloransulam-Methyl, Fenoxaprop, Fomesafen, Fluazifop, Glyphosate, Imazamox, Imazaquin, Imazethapyr, (S-) Metolachlor, Metribuzin, Pendimethalin, Tepraloxydim, Glufosinate; Soybean Insecticides: Lambda-cyhalothrin, Methomyl, Imidacloprid, Clothianidin, Thiamethoxam, Thiacloprid, Acetamiprid, Dinetofuran, Flubendiamide, Rynaxypyr, Cyazypyr, Spinosad, Spinotoram, Emamectin-Benzoate, Fipronil, Ethiprole, Deltamethrin, 13-Cyfluthrin, gamma and lambda Cyhalothrin, 4-[[(6-Chlorpyridin-3-yl)methyl](2,2-difluorethyl)amino]furan-2(5H)-on, Spirotetramat, Spinodiclofen, Triflumuron, Flonicamid, Thiodicarb, beta-Cyfluthrin; Soybean Fungicides: Azoxystrobin, Bixafen, Boscalid, Carbendazim, Chlorothalonil, Copper, Cyproconazole, Difenoconazole, Dimoxystrobin, Epoxiconazole, Fluazinam, Fluopyram, Fluoxastrobin, Flutriafol, Fluxapyroxad, Isopyrazam, Iprodione, Isotianil, Mancozeb, Maneb, Metconazole, Metominostrobin, Myclobutanil, Penthiopyrad, Picoxystrobin, Propiconazole, Propineb, Prothioconazole, Pyraclostrobin, Tebuconazole, Tetraconazole, Thiophanate-methyl, Trifloxystrobin; Sugar beet Herbicides: Chloridazon, Desmedipham, Ethofumesate, Phenmedipham, Triallate, Clopyralid, Fluazifop, Lenacil, Metamitron, Quinmerac, Cycloxydim, Triflusulfuron, Tepraloxydim, Quizalofop; Sugar beet Insecticides: Imidacloprid, Clothianidin, Thiamethoxam, Thiacloprid, Acetamiprid, Dinetofuran, Deltamethrin, B-Cyfluthrin, gamma/lambda Cyhalothrin, 4-[[(6-Chlorpyridin-3-yl)methyl](2,2-difluorethyl)amino]furan-2(5H)-on, Tefluthrin, Rynaxypyr, Cyaxypyr, Fipronil, Carbofuran; Canola Herbicides: Clopyralid, Diclofop, Fluazifop, Glufosinate, Glyphosate, Metazachlor, Trifluralin Ethametsulfuron, Quinmerac, Quizalofop, Clethodim, Tepraloxydim; Canola Fungicides: Azoxystrobin, Bixafen, Boscalid, Carbendazim, Cyproconazole, Difenoconazole, Dimoxystrobin, Epoxiconazole, Fluazinam, Fluopyram, Fluoxastrobin, Flusilazole, Fluxapyroxad, Iprodione, Isopyrazam, Mepiquat-chloride, Metconazole, Metominostrobin, Paclobutrazole, Penthiopyrad., Picoxystrobin, Prochloraz, Prothioconazole, Pyraclostrobin, Tebuconazole, Thiophanate-methyl, Trifloxystrobin, Vinclozolin; Canola Insecticides: Carbofuran, Thiacloprid, Deltamethrin, Imidacloprid, Clothianidin, Thiamethoxam, Acetamiprid, Dinetofuran, 13-Cyfluthrin, gamma and lambda Cyhalothrin, tau-Fluvaleriate, Ethiprole, Spinosad, Spinotoram, Flubendiamide, Rynaxypyr, Cyazypyr, 4-[[(6-Chlorpyridin-3-yl)methyl](2,2-difluorethyl)amino]furan-2(5H)-on.
The following examples are offered by way of illustration and not by way of limitation.

### EXPERIMENTAL EXAMPLES

### Example 1. Identification of MeSH Sequences

Integral membrane proteins play important roles within plant cells. There are many membranes and organelles within the plant cell that include but are not limited to: the chloroplast outer membrane, chloroplast inner membrane, chloroplast thylakoid membrane, mitochondrial outer membrane, mitochondrial inner membrane, endoplasmic reticulum membrane, endosome membrane, Golgi membrane, nuclear membrane, peroxisome membrane, plasma membrane, pre-lytic vacuolar membrane, protein storage vacuolar membrane, and vesicle membrane. Most membrane proteins that are targeted to these specific locations in the cell are nuclear encoded. A targeting peptide or signal sequence is typically required to direct each protein to its destination. Once the membrane protein is transported to the intended organelle, additional signal sequences may be required to further direct to internal membranes or sub-compartments. For instance, chloroplast integral membrane proteins can stop at the outer membrane, continue moving to and stop at the inner membrane, or continue to proceed all the way to the thylakoid membrane. Froehlich and Keegstra demonstrated that for a simple chloroplast integral membrane protein with a single transmembrane domain (TMD), this TMD is the signal that determines which membrane the protein will localize to once in the chloroplast. More importantly, Froehlich and Keegstra showed that proteins such as arch (accumulation and replication of chloroplasts 6), an inner membrane cell division protein, and stn8 (state transition protein kinase 8), a thylakoid membrane kinase, can be directed to new compartments simply by changing the TMD (Froehlich, J. E and K. Keegstra (2011) The Plant 1 68:844-856; Rolland et al. (2017)1. of Exp. Bot. 68:5013-5016). Aspects of the invention, name membrane-specific hydrophobic (MeSH) signal sequence as the TMD sequence(s) required to target the integral membrane protein to its intended plant membrane (e.g. MeSH protein sequences as depicted in SEQ ID Nos: 1-88 and 288). These MeSH sequences can be used to localize and fix a protein into a relative membrane via polytopic, bitopic or monotopic positioning (see Allen et al. Trends in Biochemical Sciences, Jan. 2019, Vol. 44, No. 1). Singhal and Fernandez demonstrated targeting more complex chloroplast integral membrane proteins with multiple TMDs requires other components other than the first (or a single) TMD for effective targeting (Singhal, R and D. E. Fernandez (2017)1 Exp. Bot. 68 (18):5029-5043; Uehara et al. (2016) Front. Plant Sci. 7:16; Rolland et al. (2016) Front. Plant Sci. 7:185; Rolland et al. (2017)1 Exp. Bot. 68 (18):5013-5016). Surprisingly, applicants found that by anchoring N-transgenes in various membranes of the plant cell using MeSH sequences, one could achieve higher N-transgene expression and/or mitigate or eliminate any negative side effects of expressing said N-transgene. Aspects of the invention contemplate that any MeSH sequence could be used in combination with an appropriate targeting sequence to facilitate the successful expression of a N-Transgene as compared to a control plant. By way of example, the MeSH sequences depicted by SEQ ID Nos: 1-83 and 288 and their respective nucleic acids encoding them (i.e. SEQ ID Nos: 84-166 and 287), can be employed in the methods and vectors described herein to facilitate higher expression of N-Transgenes and/or expression of N-Transgenes with decreased negative phenotypes as compared to a control plant not comprising the relative construct with MeSH sequences.

### Example 2. Identification of N-transgenes

It is a common obstacle in the molecular expression of transgenes, that in some cases the transgene has low expression and/or may lead to negative phenotypes in plants, cells and other organisms once expressed. For plants, these negative phenotypes might include, reduced or stunted growth, cell death in certain tissue types, reduced chloroplasts and/or chlorophyll, divergent morphologies, decreased number of successful transformants, etc. The following examples are focused on certain insect resistance genes that fit the definition of a N-Transgene as defined herein. It is fully contemplated that one could also use the vectors and methods taught herein to remedy situations where the N-transgene is any plant transgene (i.e. yield trait, output traits, fungal resistance, etc.). Not to be limited by theory, this could also be the case for other organisms as well. The specific N-transgene and its' function is not believed to be critical as the important aspect of the invention is that the N-transgene is bound in the respective membrane.

By way of example, members of the following insect resistance gene classes have demonstrated phytotoxicity, negative phenotypes and/or lowered expression in various crop species. The data and observations presented below are representative of results seen with these genes regardless of construct configuration, organelle targeting, or promoter selection. Applicants use in this example a fast and effective transient leaf assay (described in Example 4) that has been found to be predictive of phytotoxicity and negative phenotypes that will be observed in stable transgenic lines expressing the respective N-transgene. It is important to note that the data and observations below from the transient assay data is indicative of data and observations noticed in stable transgenic plants expressing the relative N-Transgene.

a) Yvgo Insect Resistance Gene

Agrobacterium Tobacco infiltration (using the methods described in Example 4) of various constructs expressing the gene Yvgo demonstrated Phytotoxicity observed on day 2 post infiltration. Phytotoxicity results in a browning of the infiltration area and correlates well with what will happen in a stable transgenic (i.e. the infiltration method is a good predictive model for how N-Transgenes will perform in a stable transgenic plant.) Severe phytotoxicity effects can be observed on day 7 post infiltration (infiltration area is brown and dried out as compared to controls displaying green healthy tissue). Specific organelle targeting to chloroplast, apoplast, ER, vacuole, thylakoid, and peroxisome failed to alleviate toxicity in tobacco transients. It was further attempted to stably transform soybean and Arabidopsis with expression cassettes of various targeting or expression profiles of Yvgo all which resulted in no recovered events after multiple rounds of transformation (data not shown).

b) MTX Insect Resistance Gene

As was observed for Yvgo above, Agrobacterium tobacco infiltration of the gene MTX displayed signs of phytotoxicity on day 2 with severity increasing as it reached day 7 (e.g. again, infiltration area is brown and dried out as compared to controls displaying green healthy tissue) As was the case with Yvgo, multiple expression scenarios and attempted transgenic expression in soybean resulted in poor transformation efficiency and a low numbers of events recovered. Bronzing of leaves and pods & “woody” stems was noticed in mature transgenics. In many events, a processive phenotype is observed where mature leaves show stronger phytotoxicity (i.e. leaf bronzing). Generation of T1 homozygous plants expressing MTX was very difficult to recover.

c) Monalysin Insect Resistance Gene

Agrobacterium Tobacco infiltration of monalysin showed similar effects as demonstrated for Yvgo and MTX above. Phytotoxicity was observed on day 2 post infiltration (infiltration area changes hue & wilting may be observed). Severe phytotoxicity observed on day 7 post infiltration wherein the infiltration area is brown and dried out. Additionally, soybean transformation resulted in similar phenotypes as shown for MTX, particularly there was a low number of transgenic events and high incidence of stunted growth for those events that were produced.

### Example 3. Anchoring Cassette Construction

For Tobacco transient and Stable Arabidopsis studies a pTR20-p-Base was used as the base vector. pTR20-p-Base contains the constitutive Ubiquitin10 promoter from Arabidopsis and 3′nos terminator from Agrobacterium for the gene of interest (“GOI”) expression cassette. The GOT CDS can be cloned in using PmeI/AscI. Also included in this vector is a HPPD gene used for stable transgenic selection. This gene is expressed using the constitutive double 35S promoter and 35S terminator.

All GOT CDS were synthesized with combinations of chloroplast targeting peptides and MeSH sequences which allows for the GOT (N-transgene) to be anchored into the chloroplast membrane (see Table 1). The GOT CDS fragment is contiguous with any targeting peptides and MeSH sequences and there are no intervening sequences.

The vector pBPTVM311 was used as a negative control vector. It contains luciferase (non-phytotoxic) with the constitutive Ubiquitin10 promoter from Arabidopsis and 3′nos terminator from Agrobacterium. It also contains a HPPD selectable marker expression cassette.

TABLE 1
Listing of N-Transgene and MeSH construct
combinations tested in Transient Assay.

| | CDS | | |
|---|---|---|---|
| | Chloroplast | | |
| | Transit Peptide | MeSH | |
| Vector | (SEQ ID NO:) | (SEQ ID NO:) | N-Transgene |
| pTR1301 | None | None | ARP812 (Monalysin) |
| pTR1302 | OtpC (167) | None | |
| pTR1307 | OtpC (167) | ftsh9 (84) | |
| pTR1308 | OtpC (167) | stn8 (85) | |
| pTR1311 | ftsh9 (168) | ftsh9 I&II (86) | |
| pTR1318 | arc6 (169) | arc6 (87) | |
| pTR1502 | None | None | ARP9298 (Yvgo) |
| pTR1503 | OtpC (167) | None | |
| pTR1504 | OtpC (167) | ftsh9 (84) | |
| pTR1505 | OtpC (167) | stn8 (85) | |
| pTR1519 | ftsh9 (168) | ftsh9 I&II (86) | |
| pTR1960 | arc6 (169) | arc6 (87) | |
| pTR1498 | None | None | ARP992 (MTX) |
| pTR1499 | OtpC (167) | None | |
| pTR1500 | OtpC (167) | ftsh9 (84) | |
| pTR1501 | OtpC (167) | stn8 (85) | |
| pTR1516 | ftsh9 (168) | ftsh9 I&II (86) | |
| pTR1959 | arc6 (169) | arc6 (87) | |

Further listing of Anchoring Cassettes and associated SEQ ID NOS that may be used for plant N-transgene expression are shown in Table 2 below Results from this experiment are discussed in Example 5 below.

TABLE 2
Listing of other potential MeSH and Targeting Signals that may
be used in plant Anchoring Cassettes for the optimal expression
of N-Transgenes(e.g. MTX, Yvgo, or Monalysin N-Transgenes)

| | | Targeting | MeSH |
|---|---|---|---|
| Cassettes | | Signal | Signal |
| for Plant | | SEQ ID NO: | SEQ ID NO: |
| Expres- | | (DNA/ | (DNA/ |
| sion | Protein Anchoring Target | Peptide) | Peptide) |
| pTR3007 | Mitochondria (Inner Membrane) | 170/230 | 88/5 |
| pTR3008 | Mitochondria (Inner Membrane) | 171/231 | 88/5 |
| pTR3009 | Mitochondria (Inner Membrane) | 172/232 | 89/6 |
| pTR3010 | Mitochondria (Inner Membrane) | 173/233 | 90/7 |
| pTR3011 | Mitochondria (Inner Membrane) | 174/234 | 91/8 |
| pTR3012 | Mitochondria (Inner Membrane) | 174/234 | 92/9 |
| pTR3013 | Mitochondria | 175/235 | 93/10 |
| pTR3014 | Mitochondria | 175/94 | 94/11 |
| pTR3015 | Mitochondria | 175/235 | 95/12 |
| pTR3016 | Vacuole | 176/236 | 96/13 |
| pTR3017 | Vacuole | 176/236 | 97/14 |
| pTR3018 | Vacuole | 177/237 | 98/15 |
| pTR3019 | Vacuole | 177/237 | 99/16 |
| pTR3020 | Vacuole | 178/238 | 100/17 |
| pTR3021 | Vacuole | 178/238 | 101/18 |
| pTR3022 | Vacuole | 179/239 | 102/19 |
| pTR3023 | Vacuole | 179/239 | 103/20 |
| pTR3024 | Vacuole | 180/240 | 104/21 |
| pTR3134 | Vacuole | 219/279 | 150/67 |
| pTR3136 | Plasma Membrane | 181/241 | 105/22 |
| pTR3026 | Plasma Membrane | 182/242 | 106/23 |
| pTR3027 | Plasma Membrane | 182/242 | 107/24 |
| pTR3028 | Plasma Membrane | 183/243 | 108/25 |
| pTR3029 | Plasma Membrane | 183/243 | 109/26 |
| pTR3030 | Plasma Membrane | 184/244 | 110/27 |
| pTR3036 | Plasma Membrane | 182/242 | 111/28 |
| pTR3037 | Peroxisome | 182/242 | 112/29 |
| pTR3038 | Peroxisome | 182/242 | 113/30 |
| pTR3039 | Peroxisome | 182/242 | 114/31 |
| pTR3040 | Peroxisome | 182/242 | 115/32 |
| pTR3041 | Peroxisome | 182/242 | 116/33 |
| pTR3042 | Peroxisome | 182/242 | 117/34 |
| pTR3043 | Peroxisome | 185/245 | 118/35 |
| pTR3044 | Peroxisome | 186/246 | 119/36 |
| PTR1500 | Chloroplast | 167/227 | 84/1 |
| PTR1501 | Chloroplast | 167/227 | 85/2 |
| PTR1516 | Chloroplast | 168/228 | 86/3 |
| PTR1959 | Chloroplast (inner membrane) | 168/229 | 87/4 |
| pTR3051 | Chloroplast (inner membrane) | 167/227 | 120/37 |
| pTR3052 | Chloroplast (inner membrane) | 167/227 | 121/38 |
| pTR3053 | Chloroplast (inner membrane) | 167/227 | 122/39 |
| pTR3054 | Chloroplast (inner membrane) | 167/227 | 123/40 |
| pTR3055 | Chloroplast (thylakoid) | 167/227 | 124/41 |
| pTR3056 | Chloroplast (thylakoid) | 167/227 | 125/42 |
| pTR3057 | Chloroplast (thylakoid) | 167/227 | 126/43 |
| pTR3058 | Chloroplast (thylakoid) | 167/227 | 127/44 |
| pTR3059 | Chloroplast (thylakoid) | 167/227 | 128/45 |
| pTR3060 | Chloroplast (thylakoid) | 167/227 | 129/46 |
| pTR3061 | Chloroplast (thylakoid) | 167/227 | 130/47 |
| pTR3062 | Chloroplast (thylakoid) | 167/227 | 131/48 |
| pTR3063 | Chloroplast (thylakoid) | 167/227 | 132/49 |
| pTR3064 | Chloroplast (thylakoid) | 167/227 | 133/50 |
| pTR3065 | Chloroplast (thylakoid) | 167/227 | 134/51 |
| pTR3066 | Chloroplast (thylakoid) | 167/227 | 135/52 |
| pTR3067 | Chloroplast (thylakoid) | 167/227 | 136/53 |
| pTR3068 | Chloroplast (thylakoid) | 167/227 | 137/54 |
| pTR3069 | Chloroplast (thylakoid) | 167/227 | 138/55 |
| pTR3070 | Chloroplast (thylakoid) | 167/227 | 139/56 |
| pTR3071 | Chloroplast (thylakoid) | 167/227 | 140/57 |
| pTR3073 | Chloroplast (thylakoid) | 167/227 | 141/58 |
| pTR3075 | Chloroplast (thylakoid) | 167/227 | 142/59 |
| pTR3076 | Chloroplast (thylakoid) | 167/227 | 143/60 |
| pTR3078 | Chloroplast (thylakoid) | 167/227 | 144/61 |
| pTR3080 | Chloroplast (thylakoid) | 167/227 | 145/62 |
| pTR3081 | Chloroplast (thylakoid) | 167/227 | 146/63 |
| pTR3082 | Chloroplast (thylakoid) | 167/227 | 147/64 |
| pTR3083 | Chloroplast | 167/227 | 148/65 |
| pTR3084 | Chloroplast (inner membrane) | 187/247 | 120/37 |
| pTR3085 | Chloroplast (inner membrane) | 188/248 | 121/38 |
| pTR3086 | Chloroplast (inner membrane) | 189/249 | 122/39 |
| pTR3087 | Chloroplast (inner membrane) | 190/250 | 123/40 |
| pTR3088 | Chloroplast (thylakoid) | 191/251 | 124/41 |
| pTR3089 | Chloroplast (thylakoid) | 192/252 | 125/42 |
| pTR3090 | Chloroplast (thylakoid) | 193/253 | 126/43 |
| pTR3091 | Chloroplast (thylakoid) | 194/254 | 127/44 |
| pTR3092 | Chloroplast (thylakoid) | 195/255 | 128/45 |
| pTR3093 | Chloroplast (thylakoid) | 196/256 | 129/46 |
| pTR3094 | Chloroplast (thylakoid) | 197/257 | 130/47 |
| pTR3095 | Chloroplast (thylakoid) | 198/258 | 131/48 |
| pTR3096 | Chloroplast (thylakoid) | 199/259 | 132/49 |
| pTR3097 | Chloroplast (thylakoid) | 200/260 | 133/50 |
| pTR3098 | Chloroplast (thylakoid) | 201/261 | 134/51 |
| pTR3099 | Chloroplast (thylakoid) | 202/263 | 135/52 |
| pTR3100 | Chloroplast (thylakoid) | 203/263 | 136/53 |
| pTR3101 | Chloroplast (thylakoid) | 204/264 | 137/54 |
| pTR3102 | Chloroplast (thylakoid) | 205/265 | 138/55 |
| pTR3103 | Chloroplast (thylakoid) | 205/265 | 139/56 |
| pTR3104 | Chloroplast (thylakoid) | 206/266 | 140/57 |
| pTR3105 | Chloroplast (thylakoid) | 206/266 | 141/58 |
| pTR3106 | Chloroplast (thylakoid) | 207/267 | 142/59 |
| pTR3108 | Chloroplast (thylakoid) | 207/267 | 143/60 |
| pTR3109 | Chloroplast (thylakoid) | 208/268 | 144/61 |
| pTR3110 | Chloroplast (thylakoid) | 208/268 | 145/62 |
| pTR3111 | Chloroplast (thylakoid) | 209/269 | 146/63 |
| pTR3112 | Chloroplast (thylakoid) | 209/269 | 147/64 |
| pTR3113 | Chloroplast (thylakoid) | 209/269 | 148/65 |
| pTR3114 | Chloroplast (thylakoid) | 210/270 | 87/4 |
| pTR3115 | Chloroplast (thylakoid) | 211/271 | 122/39 |
| pTR3116 | Chloroplast | 168/228 | 148/65 |
| pTR3117 | Chloroplast (inner membrane) | 169/229 | 87/4 |
| pTR3118 | Chloroplast (inner membrane) | 169/229 | 122/39 |
| pTR3119 | Chloroplast (thylakoid) | 167/227 | 130/47 |
| pTR3121 | Chloroplast (thylakoid) | 167/227 | 147/64 |
| pTR3122 | Chloroplast (thylakoid) | 167/227 | 135/52 |
| pTR3123 | Chloroplast | 167/227 | 84/1 |
| pTR3124 | Chloroplast | 167/227 | 84/1 |
| pTR3125 | Chloroplast | 167/227 | 148/65 |
| pTR3127 | Chloroplast (thylakoid) | 212/272 | 149/66 |
| pTR3137 | Chloroplast (outer membrane) | 220/280 | 151/68 |
| pTR3139 | Chloroplast (outer membrane) | 221/281 | 151/68 |
| pTR3140 | Chloroplast (thylakoid) | 222/282 | 152/69 |
| pTR3145 | Chloroplast (thylakoid) | 167/227 | 156/73 |
| pTR3146 | Chloroplast (thylakoid) | 167/227 | 157/74 |
| pTR3147 | Chloroplast (thylakoid) | 167/227 | 158/75 |
| pTR3148 | Chloroplast (thylakoid) | 167/227 | 159/76 |
| pTR3149 | Chloroplast (thylakoid) | 167/227 | 160/77 |
| pTR3150 | Chloroplast (thylakoid) | 167/227 | 160/77 |
| pTR3151 | Chloroplast (thylakoid) | 167/227 | 161/78 |
| pTR3152 | Chloroplast (thylakoid) | 167/227 | 162/79 |
| pTR3153 | Chloroplast (thylakoid) | 167/227 | 163/80 |
| pTR3154 | Chloroplast (thylakoid) | 167/227 | 164/81 |
| pTR3155 | Chloroplast (thylakoid) | 167/227 | 165/82 |
| pTR3156 | Chloroplast (thylakoid) | 167/227 | 166/83 |
| pTR3157 | Chloroplast (thylakoid) | 167/227 | 166/83 |
| pTR3158 | Chloroplast (thylakoid) | 212/272 | 161/78 |
| PTR1308 | Chloroplast | 167/227 | 287/288 |

### Example 4. Transient Assay for measuring Phytotoxic effect

Early detection of a trait gene's phytotoxicity potential is desirable for cost effective transgenic research. The phenotypic phytotoxicity evaluation is commonly done through stable plant transformation judging from the number of transgenic plants generated or the health and physical appearance of transgenic plants. In general, the assessment of phenotype in soybean can take up to ˜4 months initiated from transforming vector DNA into plants and subsequently monitoring transformation efficiency and/or plant health in the greenhouse. To speed up this process and increase the throughput, a tobacco transient infiltration method has been adapted to effectively evaluate a traits' potential phytotoxicity in tobacco plants, with the same binary vectors used for soybean stable transformation. With this adapted method, one is now able to screen a large number of vectors for the gene of interest phytotoxicity potentials within a short timeline and at reduced cost. The method allows one to quickly screen all constructs and/or traits for any negative phenotypic impact. The method also allows one to better select which construct(s) it will use in the development of commercial lines or further stable transgenic evaluations. The method can be used to predict negative phenotypes in any plant such as dicots or monocots.

The following transient assay method has been developed to assess gene expression transiently through agroinfiltration of plant tissue. The assay can be modified to use any plant tissue but, in this instance, applicants chose tobacco leaves (e.g. Nicotiana benthamiana)due to its ease of infiltration and growth. This method was surprisingly found to be a very accurate predictor for the identification of N-Transgenes and their impact on plant growth or phenotype. The method comprises the following steps. First a Agrobacterium cell suspension is prepared. This is accomplished by streaking Agrobacterium cells transformed with the binary vector of interest on selective plates and incubating at 28° C. for 1-2 days until heavy cell growth appears on the plates. Next, the Agrobacterium cells are re-suspended in co-cultivation media (CCM from PGE) with a acetosyringone at OD 600 nm of 0.3-0.5. These cells are incubated at room temperature for at least 30 minutes before proceeding to agroinfiltration. Second, N. benthaminaa plants are prepared for agroinfiltration. N. benthamiana seedlings are grown from seed in a growth chamber ((28° C./15 hours light: 26° C./9 hours dark) for approximately 1 week. The seedlings are next transferred into individual post and allowed to grow for another 2-3 weeks until 5-8 fully expanded leaves appear. Third, agroinfiltration is carried out for each of the vectors tested as well as controls. Two fully expanded leaves of a tobacco plant are used for every respective vector tested. Infiltration is carried out with a lml syringe without a needle by pushing the Agrobacterium suspension into the leaf airspaces on the underside of the respective leaf. The agroinfiltration site is marked and the plants are placed back into the growth chamber for 2 days. Finally, each of the plants (including controls) are observed for phenotypic observation using the scale described below and samples are taken to confirm transient expression via PCR of the relevant gene being tested. Sample leaf punches are taken from the area of infiltration marked in the previous step. The phenotype is supported with the molecular analysis to understand and correlate the traits phenotype with the expression data.

The following rating scale was developed to measure observed phytotoxic effect on tobacco leaves transiently expressing relevant constructs:

Transient Phytotoxicity Rating Scale (Phyto Rating)
0—No observable phytotoxicity (Completely healthy)
0/1—No or mild phytotoxicity (Slight yellowing and ambiguous based on yellowing in control)
1—Mild phytotoxicity (clear yellowing vs. control)
2—Moderate phytotoxicity (in between 1 and 3)
3—catastrophic phytotoxicity (Catastrophic phytotoxic effects by day 7)

### Example 5—Transient Assay Results for N-Transgenes Anchored in Chromosome Membranes

Table 3 summarizes the data from the transient experiments evaluating the effectiveness of anchoring known N-Transgenes (Yvgo, MTX and Monalysin) into chloroplast membranes by use of MeSH sequences and targeting peptide combinations. The results indicate that chloroplast targeting alone is insufficient to alleviate or reduce the phytotoxicity of the gene examples. In all examples where N-Transgenes were operably linked to a MeSH sequence, phytotoxic impact was decreased significantly. When otpC (chloroplast transit peptide) is used in conjunction with ftsh9 or snt8, a marked improvement is noted in phytotoxicity profiles of all tested gene classes. Furthermore, when ftsh9(I)+ftsh9(II) or arch are tested with their native transit peptides, a marked improvement is also noted in phytotoxicity profiles of all tested insect resistant gene classes.

TABLE 3
Summary of Transient Assay Data

| | CDS | | | |
|---|---|---|---|---|
| | Chloroplast | | | |
| | Target Peptide | MeSH | | Phyto |
| Vector | (SEQ ID NO:) | (SEQ ID NO:) | Gene | Rating |
| pTR1301 | N/A | N/A | ARP812 | 3 |
| pTR1302 | OtpC (167) | N/A | (Monolysin) | 3 |
| pTR1307 | OtpC (167) | ftsh9 (84) | | 0/1 |
| pTR1308 | OtpC (167) | stn8 (85) | | 0/1 |
| pTR1311 | ftsh9 (168) | ftsh9 I + II (86) | | 2 |
| pTR1318 | arc6 (169) | arc6 (87) | | 0/1 |
| pTR1502 | N/A | N/A | ARP9298 | 3 |
| pTR1503 | OtpC (167) | N/A | (YVGO) | 2 |
| pTR1504 | OtpC (167) | ftsh9 (84) | | 1 |
| pTR1505 | OtpC (167) | stn8 (85) | | 0/1 |
| pTR1519 | ftsh9 (168) | ftsh9 I + II (86) | | 1 |
| pTR1960 | arc6 (169) | arc6 (87) | | 2 |
| pTR1498 | N/A | N/A | ARP992 | 3 |
| pTR1499 | OtpC (167) | N/A | (MTX) | 3 |
| pTR1500 | OtpC (167) | ftsh9 (84) | | 0/1 |
| pTR1501 | OtpC (167) | stn8 (85) | | 0/1 |
| pTR1516 | ftsh9 (168) | ftsh9 I + II (86) | | 0/1 |
| pTR1959 | arc6 (169) | arc6 (87) | | 2 |

### Example 5. Stable T0 Arabidopsis Transformants

Transgenic Arabidopsis plants were created comprising the following expression cassettes as shown in Table 4. As summarized by Table 4, the YvgO N-Transgene ARP5561 coupled with MeSH sequences produced more events and a higher ratio of expressing events than control plants comprising only a chloroplast targeting peptide. In regard to the Monalysins, MeSH sequences improved performance. For the ARP812 gene, expression cassettes comprising ftsh9 or stn8 MeSH sequences resulted in viable transplants with normal phenotype and seed set. Interestingly the Monalysin gene ARP2040 control plant did result in expressing transplants and at a higher number than the ARP2040 cassettes comprising ftsh9 or stn8. However, as the control plants matured negative phenotypes and seed infertility became an issue which was not noticed for ARP2040 cassettes comprising the relative MeSH sequences.

In regard to MTX, ARP4204 coupled with MeSH sequences produced more events and a higher ratio of expressing events than control plants comprising only a chloroplast targeting peptide. As demonstrated in a second experiment, ARP4204+Snt8 2.9 resulted an even higher proportion of expressing events as compared to ARP4204+Stn8.

In conclusion, all case scenarios using ftsh9 (84) and stn8 (85 & 166) MeSH sequences showed a decrease in presence of negative phenotypes and/or resulted in plants capable of producing viable fertile seed. MeSH arc6 (87) with its native Chloroplast Targeting sequence arc6 (169) and MeSH ftsh9 I&II (86) with its native Chloroplast Targeting sequence ftsh (168) showed a decrease of negative phenotype and/or resulted in plants capable of producing viable fertile seed with some N-transgenes.

TABLE 4
Stable Transgenic Arabidopsis Experiment

| | CDS | | | | |
|---|---|---|---|---|---|
| | Chloroplast | | | | Percent |
| | Targeting Peptide | MeSH | N-Transgene | # Plants | Expression |
| Vector | (SEQ ID NO:) | (SEQ ID NO:) | (Type) | Generated | Rate * |
| PTR2015 | OtpC (167) | N/A | ARP9299 (YvgO) | 0 | 0 |
| PTR2016 | arc6 (169) | stn8 (85) | ARP9299 (YvgO) | 6 | 50 |
| PTR2017 | ftsh (168) | arc6 (87) | ARP9299 (YvgO) | 12 | 0 |
| PTR2018 | OtpC (167) | ftsh9 I&II (86) | ARP812 (Monalysin) | 2 | 100 |
| PTR2019 | OtpC (167) | ftsh9 (84) | ARP812 (Monalysin) | 37 | 53 |
| PTR2020 | OtpC (167) | stn8 (85) | ARP812 (Monalysin) | 4 | 50 |
| PTR2021 | OtpC (167) | N/A | ARP814 (Monalysin) | 20 | 100** |
| PTR2022 | OtpC (167) | ftsh9 (84) | ARP814 (Monalysin) | 40 | 7** |
| PTR2023 | OtpC (167) | stn8 (85) | ARP814 (Monalysin) | 29 | 53** |
| PTR2024 | OtpC (167) | stn8 (85) | ARP9299 (YvgO | 28 | 73 |
| PTR2025 | OtpC (167) | Stn8 2.9 (287) | ARP9299 (YvgO | 23 | 92 |

* Percent Expression Rate is the of number of plants with confirmed Western Blot expression of respective N-Transgene divided by the total number of events sampled
**ARP2040, produced more plants in the absence of MeSH sequences initially however, as these plants matured there was a high incidence of negative phenotypes and seed infertility in the control group. For the lines comprising ftsh9 and stn8 MeSH sequences, no negative phenotypes or seed infertility were observed in respective matured lines

### Example 6. Soybean Transformation

Soybean transformation is achieved using methods well known in the art, such as the one described using the Agrobacterium tumefaciens mediated transformation soybean half-seed explants using essentially the method described by Paz et al. (2006), Plant cell Rep. 25:206. Transformants are identified using tembotrione as selection marker. The appearance of green shoots was observed, and documented as an indicator of tolerance to the herbicide isoxaflutole or tembotrione. The tolerant transgenic shoots will show normal greening comparable to wild-type soybean shoots not treated with isoxaflutole or tembotrione, whereas wild-type soybean shoots treated with the same amount of isoxaflutole or tembotrione will be entirely bleached. This indicates that the presence of the HPPD protein enables the tolerance to HPPD inhibitor herbicides, like isoxaflutole or tembotrione.

Tolerant green shoots are transferred to rooting media or grafted. Rooted plantlets are transferred to the greenhouse after an acclimation period. Plants containing the transgene are then sprayed with HPPD inhibitor herbicides, as for example with tembotrione at a rate of 100 g AI/ha or with mesotrione at a rate of 300 g AI/ha supplemented with ammonium sulfate methyl ester rapeseed oil. Ten days after the application the symptoms due to the application of the herbicide are evaluated and compared to the symptoms observed on wild type plants under the same conditions.

### Example 7: Stable T0 Soybean Event Generation and Lepidopteran Feeding Assays

Three vectors were transformed. ARP992 (MTX) with chloroplast targeting with and without MeSH stn8-2.9 (166). In addition, a plant selectable marker only vector was incorporated as a control. Incorporation of the MeSH stn8-2.9 (166) resulted in more transformants and importantly more expressing transformants. Lepidopteran efficacy was observed against Anticarsia gemmatalis. Results are summarized in Table 5 below.

TABLE 5
Evaluation of T0 Soybean Events Comprising N-Transgene With and Without N-Transgene

| | | | | Expression | | | |
|---|---|---|---|---|---|---|---|
| Cassette | | | | # Copy | | | |
| | Chloroplast | | | number | | Insect Bioassay | |
| | Target | | | positive | WB+ | Events | Mean leaf disk |
| Vector | Peptide | MeSH | Gene | events | % | screened | damage area |
| pBas04410 | OtpC (167) | None | ARP992 (MTX) | 17 | 6% | N/A | |
| pBas04412 | OtpC (167) | stn8-2.9 (287) | ARP992 (MTX) | 37 | 43% | 10 | 67.6% +/− 20% |
| pBas04413 | N/A | N/A | Control | 82 | N/A | 25 | 93.1% +/− 6% |

### Example 7: Cotton T0 plant establishment and selection

Cotton transformation is achieved using methods well known in the art, especially preferred method in the one described in the PCT patent publication WO 00/71733. Regenerated plants are transferred to the greenhouse. Following an acclimation period, sufficiently grown plants are sprayed with HPPD inhibitor herbicides as for example tembotrione equivalent to 100 or 200 gAI/ha supplemented with ammonium sulfate and methyl ester rapeseed oil. Seven days after the spray application, the symptoms due to the treatment with the herbicide are evaluated and compared to the symptoms observed on wild type cotton plants subjected to the same treatment under the same conditions.

### Example 8. Transformation of Maize Cells

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 mL/L (of 1000x Stock) N6 Vitamins; 800 mg/L L-Asparagine; 100 mg/L Myo-inositol; 1.4 g/L L-Proline; 100 mg/L Casamino acids; 50 g/L sucrose; 1 mL/L (of 1 mg/mL Stock) 2,4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to the genes of the invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

### Materials

DN62A5S Media

| Components | Per Liter | | Source |
|---|---|---|---|
| Chu's N6 Basal | 3.98 | g/L | Phytotechnology Labs |
| Salt Mixture | | | |
| (Prod. No. C 416) | | | |
| Chu's N6 Vitamin | 1 mL/L (of 1000x Stock) | | Phytotechnology Labs |
| Solution | | | |
| (Prod. No. C 149) | | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |

| | | |
|---|---|---|
| 2,4-D | 1 mL/L (of 1 mg/mL Stock) | Sigma |
| (Prod. No. D-7299) | | |

The pH of the solution is adjusted to pH 5.8 with 1N KOH/1N KCl, Gelrite (Sigma) is added at a concentration up to 3 g/L, and the media is autoclaved. After cooling to 50° C., 2 ml/L of a 5 mg/ml stock solution of silver nitrate (Phytotechnology Labs) is added.

### Example 9. Transformation of Anchoring Cassettes of the Invention in Plant Cells by Agrobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for about 5-10 min, and then plated onto co-cultivation media for about 3 days (22° C. in the dark). After co-cultivation, explants are transferred to recovery period media for 5-10 days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art.

### Example 10. Transformation of Rice

Immature rice seeds, containing embryos at the right developmental stage, are collected from donor plants grown under well controlled conditions in the greenhouse. After sterilization of the seeds, immature embryos are excised and pre-induced on a solid medium for 3 days. After pre-induction, embryos are immersed for several minutes in a suspension of Agrobacterium harboring the desired vectors. Then embryos are co-cultivated on a solid medium containing acetosyringone and incubated in the dark for 4 days. Explants are then transferred to a first selective medium containing phosphinotricin as selective agent. After approximately 3 weeks, scutella with calli developing were cut into several smaller pieces and transferred to the same selective medium. Subsequent subcultures are performed approximately every 2 weeks. Upon each subculture, actively growing calli are cut into smaller pieces and incubated on a second selective medium. After several weeks calli clearly resistant to phosphinotricin are transferred to a selective regeneration medium. Plantlets generated are cultured on half strength MS for full elongation. The plants are eventually transferred to soil and grown in the greenhouse.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method of increasing expression levels or reducing negative impact of a N-transgene in a plant, wherein said method comprises the steps consisting of:
a) operably linking a MeSH DNA sequence and a promoter to a N-transgene therefore creating an Anchoring Cassette, wherein when said Anchoring Cassette is expressed in a plant cell, it produces a protein that anchors into an Anchoring Membrane;
b) transformation of the Anchoring Cassette of a) into a plant cell; and
c) therefore expressing a N-transgene at increased levels of transcript or protein, or having a reduced incident of negative phenotype as compared to a control plant that does not anchor the relevant protein into a cellular membrane.

2. The method of claim 1, wherein the MeSH DNA sequence is any one of SEQ ID Nos: 84-166; 287 and 289-290.

3. The method of claim 1, wherein the N-transgene is a Pesticidal N-Transgene.

4. The method of claim 3, wherein the Pesticidal N-Transgene is active against any one of Lepidopteran, Hemipteran, or Coleopteran insects.

5. The method of claim 4, wherein the N-Transgene is a MTX, YVGO or Monolysin Pesticidal N-Transgene.

6. The method of claim 1, wherein the N-transgene is a gene that confers in a transgenic plant resistance against a plant disease or fungus.

7. The method of claim 1, wherein the N-transgene is a gene that confers in a transgenic plant increased yield or abiotic stress tolerance, modified growth or flowering time or increased tolerance to biotic stress.

8. The method of claim 1, wherein the promoter, MeSH DNA sequence and N-Transgene are operably linked to a 5′ target signal sequence.

9. The method of claim 8, wherein the target signal targets the protein encoded by the N-transgene to any one of: the chloroplast outer membrane, chloroplast inner membrane, chloroplast thylakoid membrane, mitochondrial outer membrane, mitochondrial inner membrane, peroxisome membrane, plasma membrane, nuclear membrane, pre-lytic vacuolar membrane, protein storage vacuolar membrane, endosome membrane, vesicle membrane, cell wall, ER membrane or Golgi membrane.

10. The method of claim 9, wherein the target signal sequence is any one of SEQ ID Nos: 167-226.

11. The method of claim 10, wherein the MeSH DNA sequence encodes a protein comprising any one of SEQ ID Nos: 1-83 or 288

12. The method of claim 1, wherein the plant is either a monocot or dicot plant.

13. The method of claim 12, wherein the plant is a soybean, maize, rice, cotton or rape seed plant.

14. The method of claim 1, wherein the promoter is selected from the group consisting of a plant constitutive promoter, a leaf preferred promoter, a root preferred promoter or a plant tissue preferred promoter.

15. A plant expression vector comprising the Anchoring Cassette of claim 1.

16. A cell comprising the Anchoring Cassette of claim 1.

17. The cell of claim 16, wherein the cell is a plant cell.

18. The cell of claim 16, wherein the cell is a bacterial or yeast cell.

19. A plant or plant part comprising the Anchoring Cassette of claim 1.

20. A plant expression vector comprising a Pesticidal N-transgene operably linked to a MeSH DNA sequence that directs the expressed Pesticidal N-transgene protein to anchor into a Anchoring Membrane.

21. The plant expression vector of claim 20, wherein the MeSH DNA sequence encodes a protein comprising any one of SEQ ID Nos: 1-83 or 288.

22. The plant expression vector of claim 20, wherein the N-Transgene is a MTX, YVGO or Monolysin Pesticidal N-Transgene.

23. A plant, plant cell, plant part, or bacterial cell comprising the vector of claim 20.

24. A method of predicting whether a transgene will be phytotoxic in a stable transgenic plant, said method comprising the steps consisting of:
a) agroinfiltration of a binary vector into a plant leaf wherein the binary vector comprises at least one plant expression cassette comprising a transgene operably linked to a plant promoter;
b) transiently express the expression cassette of a) in said plant leaf;
c) evaluate leaf agroinfiltration site over course of days for signs of phytotoxicity;
d) therefore, predicting whether said transgene will be phytotoxic in a stable transgenic plant expressing said transgene.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
